(19) United States
(12) Patent Application Publication
(10) Pub. No.: US 2020/0085348 A1
(43) Pub. Date: Mar. 19, 2020

(54) SIMULATION OF PHYSIOLOGICAL FUNCTIONS FOR MONITORING AND EVALUATION OF BODILY STRENGTH AND FLEXIBILITY

(71) Applicant: L & C Orthopedics, LLC, Mendon, NY (US)

(72) Inventors: Jeffrey S. Lillie, Mendon, NY (US); Daniel A. Chapin, Mendon, NY (US); Brittany E. Lillie, San Diego, CA (US); Kahl Goldfarb, San Diego, CA (US)

(21) Appl. No.: 16/576,657

(22) Filed: Sep. 19, 2019

Related U.S. Application Data
(63) Continuation of application No. 15/229,225, filed on Aug. 5, 2016, now abandoned.
Application No. 15/229,225 is a continuation of application No. 15/214,686, filed on Jul. 20, 2016, now abandoned.

(51) Int. Cl. A61B 5/11; A61B 5/00; A61B 5/22; G16H 50/50

(52) U.S. Cl. CPC A61B 5/1128; A61B 5/1121; A61B 5/7246; A61B 5/0077; A61B 5/224; A61B 5/4585; A61B 2576/00; A61B 5/7275; A61B 5/4533; G16H 50/50; A61B 5/4528; A61B 2503/10; A61B 2505/09; A61B 5/486

(57) ABSTRACT

The simulation of physiological functions for monitoring and evaluation of bodily strength and flexibility includes a method and system for automated biomechanical analysis for simulated bodily joint movements. In one use, the simulation supports anterior cruciate ligament (ACL) injury prevention and recovery. The disclosure includes processing an automated and simulated biometric analysis algorithm on a computer processor and recording measurements relating to a plurality of physical therapy tests. The disclosure tracks the movement of joints on the subject's body from the subject's trunk downward to the subject's toes and calculates the results of the physical therapy tests for analyzing angles, movement quality, and related interrelationships amongst said joints. The method and system further derive and provide reports of comparisons of results with normative values and associate indicators for the comparisons to the potential of the subject to experience biomechanical conditions of injury or development.

### CROSS REFERENCE TO RELATED APPLICATIONS

This application further claims the benefit of the following non-provisional application, all of which is here expressly incorporated by reference:

Ser. No. 15/229,225 entitled “METHOD AND SYSTEM FOR AUTOMATED BIOMECHANICAL ANALYSIS OF BODILY STRENGTH AND FLEXIBILITY,” filed on Aug. 5, 2016 with Attorney Docket No. LCOR001US0TR.

### FIELD OF THE INVENTION

The present disclosure relates to simulation and evaluation of human physiological measurement, analysis, and diagnosis and, more particularly to a method and system for simulation of physiological functions for monitoring and evaluation of bodily strength and flexibility automated biomechanical analysis of bodily strength and flexibility. Additionally, the present disclosure provides a method and system for simulation and evaluation of biomechanical functions for predicting, measuring, and diagnosing anterior cruciate ligament (ACL) symptoms, as well as other bodily joints or locations, for example, the lower back, shoulder, and elbow.

### BACKGROUND OF THE INVENTION

Biomechanics is the study of human motion. The simulation and study of biomechanics is important when determining what causes injuries and therefore how we can prevent them re-occurring. This is especially important in elite athletes but can be a major cause in recurrent injuries in the less gifted amateur athlete. Physiotherapists are professionally trained to detect biomechanical faults which can predispose you to injury.

Biomechanical simulation and functional analysis can involve: (a) gait analysis—study of your walking pattern; (b) running analysis—study of your running style; (c) video analysis or motion capture analysis; (d) sports biomechanics—sport specific analysis; (e) workplace analysis—study of how you do you job and (e) biomechanics of running, sprinting, swimming, throwing etc. One area of particular interest in biomechanical analysis is the assessment of problems associated with the anterior cruciate ligament or ACL.

The ACL is one of a pair of cruciate ligaments (the other being the posterior cruciate ligament) in the human knee. They are also called cruciform ligaments as they are arranged in a crossed formation. In the quadruped stifle joint (analogous to the knee), based on its anatomical position, it is also referred to as the cranial cruciate ligament. The anterior cruciate ligament is one of the four main ligaments of the knee, and the ACL provides 85% of the restraining force to anterior tibial displacement at 30 degrees and 90 degrees of knee flexion.

The ACL originates from deep within the notch of the distal femur. Its proximal fibers fan out along the medial wall of the lateral femoral condyle. There are two bundles of the ACL—the anteromedial and the posterolateral, named according to where the bundles insert into the tibial plateau. (The tibia plateau is a critical weight-bearing region on the upper extremity of the tibia). The ACL attaches in front of the intercondyloid eminence of the tibia, being blended with the anterior horn of the medial meniscus. These attachments allow the ACL to resist anterior translation and medial rotation of the tibia, in relation to the femur.

ACL tears are one of the most common knee injuries, with over 100,000 ACL tears in the US occurring annually. Most ACL tears are a result of landing or planting in cutting or pivoting sports, with or without contact. Most serious athletes will require an ACL reconstruction if they have a complete tear and want to return to sports, because the ACL is crucial for stabilizing the knee when turning or planting. The surgeon will make holes in the patient's bones to run the tissue through, and the tissue serves as the patient's new ACL. Recovery time ranges between 1-2 years or longer.

Tearing the anterior cruciate ligament can sometimes be part of a knee injury known as “the terrible triad”. This consists of the simultaneous tearing of the anterior cruciate ligament (ACL), medial collateral ligament (MCL), and medial meniscus.

The ACL can be treated non-operatively with strengthening and rehabilitation and occasionally injections when the ACL is not completely torn and the knee is still stable or if the patient is not doing activities requiring cutting and pivoting or similar actions. The mainstay of ACL non-operative treatment is strengthening of the muscles around the knee, especially the hamstrings. Focused physical therapy supervised by an orthopedic specialist can be an effective way to accomplish this.

Because of the seriousness and lengthy recovery time for ACL injuries, there is the need for new insights in ACL injury prevention. Historically, ACL injury prevention has followed a series of flexibility and strengthening exercises. Screening was typically done using time intensive measurements, observations and recorded notes. None of this information was typically kept in a form that would allow for scientists, medical professionals, physicians assistants and physical therapists to look for trends across populations.

Moreover, historically repeating measures over time has been difficult, if not impossible, without a significant amount of subjective and inaccurate information. This has been due to the generally inability to make accurate objective measurements of a patient's bodily strength and joint flexibility.

At a very high level, doctors and physical therapists evaluate patients or athletes principally through subjective measures. Because most data is subjective, re-evaluation of patients and athletes is subjective and generally inexact. This makes assessments across a population to look for risk of injury difficult, if not impossible.

Importantly, there is a need for a method and system for allowing the measurement and assessment of an individual patient's progress or change in bodily strength and flexibility over time. There is the need for allowing a medical professional the ability to grade for such measurements across a team or like population.

From such measurements, there is the need to identify injuries that occur and trends in measured weakness. For example, where a number of ACL injuries may occur in a high school athletic team during a season, there is the need for a method and system that will allow an objective look at the profiles of the athletes and see what other athletes may be at risk.

There is a further need for ways to accelerate ACL injury recovery.

A further need exists for improving significantly the understanding and accuracy of measures ACL screening.

In light of the aforementioned limitations and concerns, there is the need for a method and system for automated biomechanical analysis of bodily strength and flexibility, and more specifically a method and system for predicting, measuring, and diagnosing ACL symptoms.

Now, a system capable of addressing ACL symptoms also may have application in a broad array of biomechanical application of value and benefit to physiotherapists. Accordingly, the scope of the present disclosure extends beyond the prediction, measurement, and diagnosing of ACL symptoms.

### BRIEF SUMMARY OF THE INVENTION

The disclosed subject matter provides for a method and system for automated biomechanical analysis of bodily strength and flexibility, and with regard to the present disclosure a method and system for predicting, measuring, and diagnosing ACL symptoms for injury prevention and recovery offering significantly improved important and accurate ACL measurements including and advanced ACL screening protocol, a 3-dimensional (3-D) camera system, point tracking methods, and novel extraction algorithms for use in an advanced digital processing system.

In light of the above, the present disclosure provides a method and system for automated biomechanical analysis that enables anterior cruciate ligament (ACL) injury prevention and recovery. The method and system provide for processing an automated ACL injury analysis algorithm on a computer processor. The method and system further provides for recording measurements relating to a plurality of physical therapy tests of a subject using a three-dimensional measurement imaging device associated with said computer processor. By recording such measurements, the present disclosure allows for tracking the movement of joints on the subject's body from the subject's trunk downward to the subject's toes for each of said plurality of physical therapy tests using the associated imaging device and computer processor. These recorded measurements are then available for the computer processor to calculate the results of said plurality of physical therapy tests using said automated ACL injury analysis algorithm. The ACL injury analysis algorithm provides instructions for the computer processor to analyze angles, movement quality, and related interrelationships amongst said joints. The disclosed subject matter further enables the computer processor to derive comparisons of the calculated results with a plurality of normative values stored on the computer process. The computer processor further may associate indicators for said comparisons. The computer process stores or otherwise accesses the associated indicators and relates the indicators to the potential of the subject for experiencing an ACL injury. These indictors and the potential for ACL injury are further made available on one or more displays associated with the computer processor.

In light of the present disclosure, here appears a method and system for providing new insights in ACL injury prevention that properly addresses the seriousness and lengthy recovery time for ACL injuries.

The subject matter of the present disclosure provides ways to accelerate ACL injury recovery. One appealing aspect of the presently disclosed inventive subject matter includes of feature of, for the first time, all joints being accurately tracked and measured through all movements. Using medical guidelines, all joint movements that fall outside of prescribed norms are quickly flagged on an easy to visualize dashboard. In addition, the present method and system provide a detailed and tailored report for use by a medical professional and/or patient that includes measured and accurate information relating to bodily strength and flexibility. Having this accurate repeatable system in place allows for progress tracking during a variety of therapy protocols.

Moreover, the presently explained and disclosed novel subject matter provides for improving significantly the understanding and accuracy of measures ACL screening. For, although work on ACL injury prevention has developed over recent decades, no-one has developed a method and system for ACL injury prevention that couples state of the art ACL screening protocol with the 3-D imaging, joint tracking and associated screening algorithms.

In essence, the present disclosure enables a method and system for automated biomechanical analysis of bodily strength and flexibility, and more specifically a method and system for predicting, measuring, and diagnosing ACL symptoms.

Moreover, the disclosed subject matter provides the technical advantage of the ability to develop and understand trends among teams, positions, and players. That is, the present method and system provide both a clinical benefit and a benefit for coaches. For example, a coach who is recruiting 2 similar athletes may select 1 over the other simply due to a higher full body assessment score. Simply because due to superior flexibility/form/strength the risk of injury is lower. The counter could be used to make sure an athlete is at the same level or improved at the start of each season.

A technical advantage of the present disclosure includes the ability to prevent ACL and related injuries, because analysis is measured and can be predictive, even prior to the onset of pain or inflammation.

The disclosed subject matter also provides for the improvement of sports performance, because of the ability measure and perceive improper joint movements, as well as to document such movements for general movement improvements while performing sports activities.

A yet further advantage of the presently disclosed subject matter includes an improved ability to conduct orthopedic studies, document orthopedic experiments, and develop an improved body of orthopedic research literature to advance related science and medical treatment.

Yet another advantage of the presently disclosed subject matter includes the ability to move beyond subjective measures of ACL injury conditions. Because the presently disclosed method and system provide for more precise and objective patient orthopedic data, re-evaluation of a patient or athlete over time becomes more precise.

A further advantage the present disclosure also includes the ability assess conditions relating to other lower extremities ligaments and tendons beyond the ACL. For example, upper extremities, such as elbow and shoulder ligaments may be measured objectively to assess conditions warranting attention.

Still further, the presently disclosed subject matter has use for analyzing a baseball swing or other sports swing or movement to determine optimal performance, as well as the likelihood of a physically detrimental of injury-promoting motion that may need modification or correction.

### BRIEF DESCRIPTION OF THE DRAWINGS

The present subject matter will now be described in detail with reference to the drawings, which are provided as illustrative examples of the subject matter so as to enable those skilled in the art to practice the subject matter. Notably, the FIGUREs and examples are not meant to limit the scope of the present subject matter to a single embodiment, but other embodiments are possible by way of interchange of some or all of the described or illustrated elements and, further, wherein:

FIG. 1 depicts a general flow diagram for the processes enabled through the present disclosure;

FIG. 2 diagrams a recurring pattern of ACL injury that may occur with an athlete without attention to proper training and exercise movements;

FIG. 3 schematically shows a computing system forming a constituent portion of the method and system of the present disclosure;

FIG. 4 shows a process flow for the method and system of the present disclosure;

FIG. 5 illustrates the identification of selected data collection nodes for an exemplary subject for the use of the disclosed subject matter;

FIG. 6 shows a substantially frontal view of an exemplary skeletal model overlaying a video image of an athlete for demonstrating use of data collection nodes;

FIGS. 7A and 7B shows how the cameras coordinate system works in terms of X, Y, and Z;

FIGS. 7C and 7D shows two different ways in which angles are formed by joints

FIG. 8A shows an image associated with video footage of the collected data points for a front view of an individual for assessing ACL related information;

FIG. 8B details an exemplary results tracking output;

FIGS. 9A, 9B, and 9C portray a computer screen interface for reporting a physical body node data derived from the 3-D camera recordings;

FIG. 10 presents graphs of collected data points relating to right knee measurements of an individual for analyzing right knee flexion and valgus/varus;

FIGS. 11A through 11E show data points for a double leg squat test enabled by the presently disclosed subject matter;

FIGS. 12A through 12D present a side display of data collection nodes overlaying a front image of an athlete for analyzing a double leg squat test according the present teachings;

FIG. 13 shows an ACL prevention data grid for indicating whether the method and system of the present disclosure has determined an ACL injury likely condition;

FIG. 14 presents data by which a clinician may study right knee flexion, valgus, and varus throughout a complete range of motion;

FIGS. 15A through 15F show variations of image screens applicable to baseball bat swinging analyses using the processes of the present disclosure; and

FIGS. 16A through 16H show variations of image variations of image screens applicable to hitting analyses using the processes of the present disclosure.

### DETAILED DESCRIPTION OF ILLUSTRATIVE EMBODIMENTS

The detailed description set forth below in connection with the appended drawings is intended as a description of exemplary embodiments in which the presently disclosed process can be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed method and system. However, it will be apparent to those skilled in the art that the presently disclosed process may be practiced without these specific details. In some instances, well-known structures and devices are shown in block diagram form in order to avoid obscuring the concepts of the presently disclosed method and system.

In the present specification, an embodiment showing a singular component should not be considered limiting. Rather, the subject matter preferably encompasses other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present subject matter encompasses present and future known equivalents to the known components referred to herein by way of illustration.

Although the method and system for predicting, measuring, and diagnosing anterior cruciate ligament (ACL) symptoms here disclosed have been described in detail herein with reference to the illustrative embodiments, it should be understood that the description is by way of example only and is not to be construed in a limiting sense. It is to be further understood, therefore, that numerous changes in the details of the embodiments of this disclosed process and additional embodiments of this method and system for automated biomechanical analysis of bodily strength and flexibility will be apparent to, and may be made by, persons of ordinary skill in the art having reference to this description. It is contemplated that all such changes and additional embodiments are within the spirit and true scope of this disclosed method and system as claimed below.

The disclosed subject matter allows a clinician to assess the risk of ACL injury in an efficient, accurate, repeatable and recordable manner. With medically approved limits set for each joint and movement, the present disclosure facilitate identifying problem movements quickly and accurately. The ability to accurately measure any joint in 3-D space and use data collection and analysis algorithms for extracting position information and presenting in useful clinical dashboard.

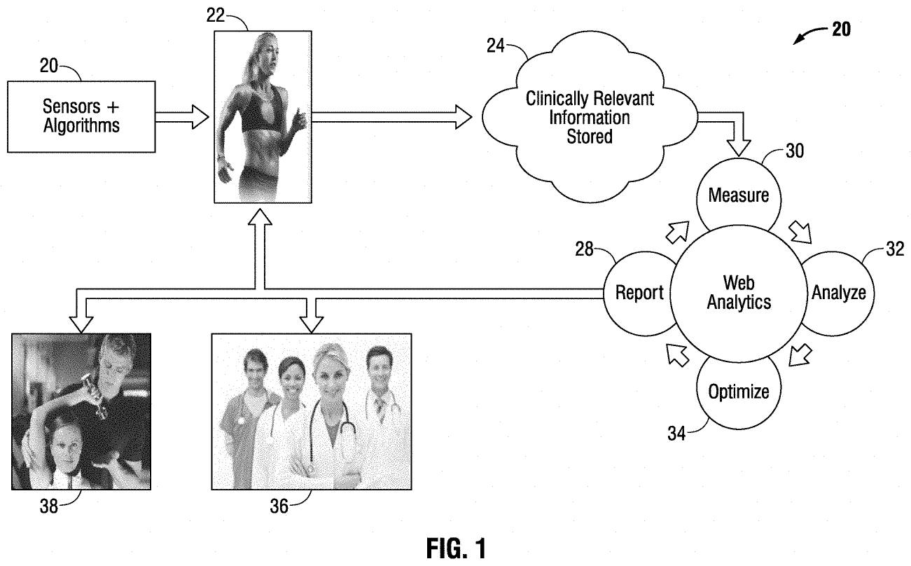

FIG. 1 depicts an overall view of automated biomechanical analysis process 10 that enables anterior cruciate ligament (ACL) injury prevention and recovery. Process 10) affords accurate physiological measurements employing the combination of a state of the art ACL screening protocol, a three-dimensional 3-D) camera, a point tracking algorithm, and proprietary extraction algorithms all operating in association with a state of the computer. Beginning with sensors functions 20 the present disclosure analyses biometric data that an athlete 22 may generate when performing sports functions that may cause ACL injuries or related problems. The data concerning athlete 22 that process 10 generates may be stored either locally or in a cloud-based environment 24 to produce clinically relevant information. From cloud-based storage 24, the present system may perform web analytics 26, for generating reports 28, measurements 30, analyses 32, and optimizations 34. Following the web analytics 26 functions, information relating to athlete 22 may transmitted to healthcare professionals 36, to the athlete 22, and/or to therapist 38.

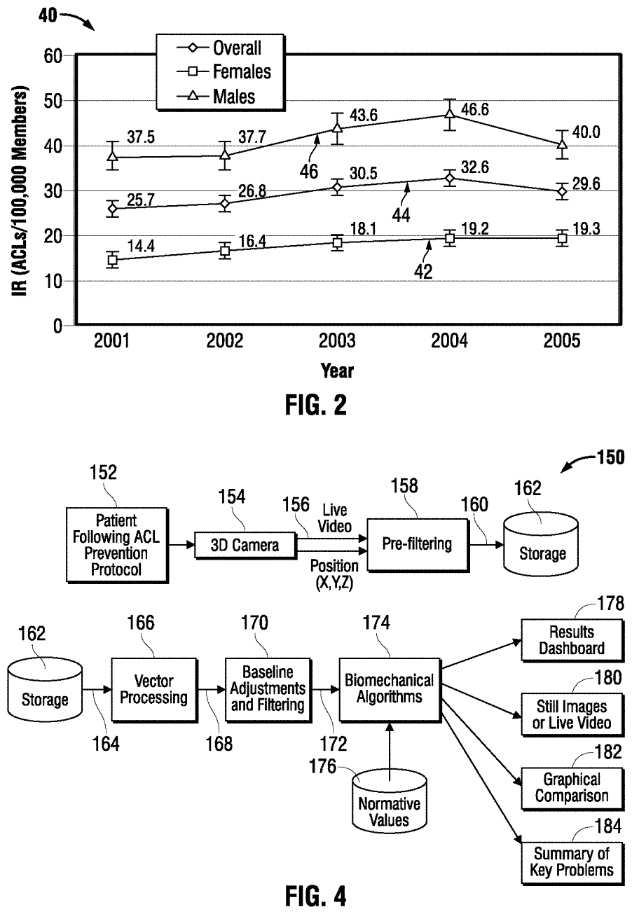

FIG. 2 provides diagram 40 which documents the recurrence of ACL injury with athletes that may arise without attention to proper training and exercise movements. Incidence rate and 95% confidence interval are plotted for ACL reconstructions per 100,000 members, where “IR” relates to the incidence rate. In particular, chart line 42 reports data for female athletes, line 44 reports data for male athletes, and line 46 reports both female and male athlete data per 100,000 members. Process 10 of the present disclosure addresses this phenomenon to provide otherwise unavailable and precise information relating to ACL injury prone conditions to identify conditions and isolate certain activities in which an athlete engage or experience that may induce ACL injuries. These may be identified well prior to the athlete sensing pain or experiencing the injurious effect of ACL tears or damage, thereby reducing or eliminating the need for ACL reconstructions.

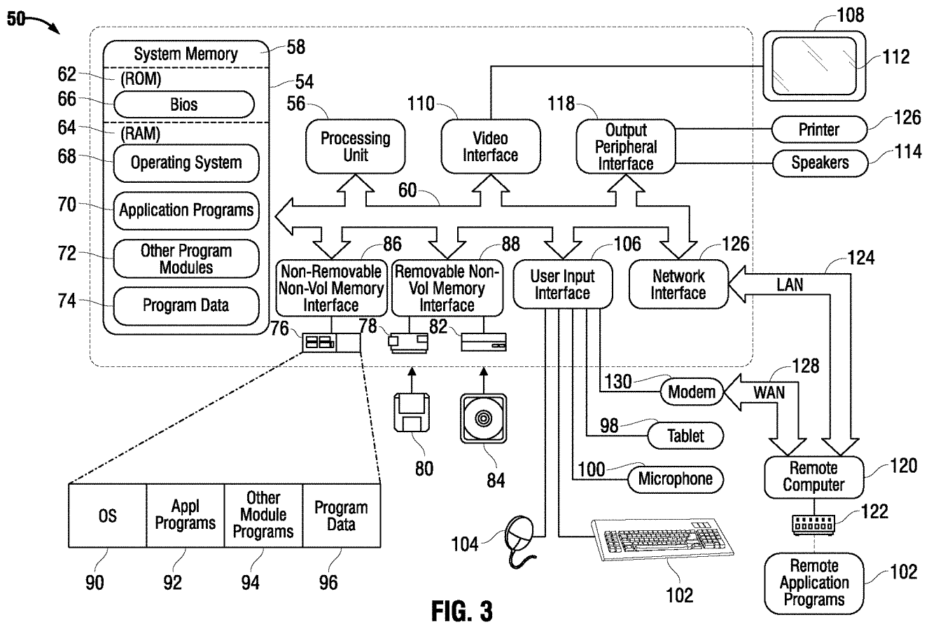

The data capture, analysis, and use of the method and system of the present disclosure require the use of a computing system associated with a three-dimensional camera system. Thus, with reference to FIG. 3, an exemplary system within computing environment 50 for implementing the disclosure includes a general purpose computing device in the form of computing system 52, commercially available from, for example, Intel, IBM, AMD, Apple, Motorola, Cyrix, etc. Components of computing system 54 may include, but are not limited to, processing unit 56, system memory 58, and system bus 60 that couples various system components including system memory 58 to processing unit 56. System bus 60 may be any of several types of bus structures including a memory bus or memory controller, a peripheral bus, or a local bus using any of a variety of bus architectures.

Computing system 52 typically includes a variety of computer readable media. Computer readable media can be any available media that can be accessed by computing system 52 and includes both volatile and nonvolatile media, and removable and non-removable media. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data.

Computer memory includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing system 52.

System memory 58 includes computer storage media in the form of volatile and/or nonvolatile memory such as read only memory (ROM) 62 and random access memory (RAM) 64. A basic input/output system (BIOS) 66, containing the basic routines that help to transfer information between elements within computing system 52, such as during start-up, is typically stored in ROM 62. RAM 64 typically contains data and/or program modules that are immediately accessible to and/or presently being operated on by processing unit 56. By way of example, and not limitation, operating system 68, application programs 70, other program modules 72, and program data 74 are shown.

Computing system 52 may also include other removable/non-removable, volatile/nonvolatile computer storage media. By way of example only, hard disk drive 76 that reads from or writes to non-removable, nonvolatile magnetic media, a magnetic disk drive 78 that reads from or writes to removable, nonvolatile magnetic disk 80, and an optical disk drive 82 that reads from or writes to removable, nonvolatile optical disk 84 such as a CD ROM or other optical media could be employed to store the invention of the present embodiment. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 76 is typically connected to the system bus 60 through a non-removable memory interface such as interface 86, and magnetic disk drive 78 and optical disk drive 82 are typically connected to the system bus 60 by a removable memory interface, such as interface 88.

The drives and their associated computer storage media, discussed above, provide storage of computer readable instructions, data structures, program modules and other data for computing system 52. For example, hard disk drive 76 is illustrated as storing operating system 90, application programs 92, other program modules 94 and program data 96. Note that these components can either be the same as or different from operating system 68, application programs 70, other program modules 72, and program data 74. Operating system 90, application programs 92, other program modules 94, and program data 96 are given different numbers here to illustrate that, at a minimum, they are different copies.

A user may enter commands and information into the computing system 52 through input devices such as tablet or electronic digitizer 98, microphone 100, keyboard 102, and pointing device 104, commonly referred to as a mouse, trackball, or touch pad. These and other input devices are often connected to the processing unit 56 through a user input interface 106 that is coupled to the system bus 60, but may be connected by other interface and bus structures, such as a parallel port, game port or a universal serial bus (USB).

Monitor 108 or other type of display device is also connected to the system bus 60 via an interface, such as a video interface 110. Monitor 108 may also be integrated with a touch-screen panel 112 or the like. Note that the monitor and/or touch screen panel can be physically coupled to a housing in which computing system 52 is incorporated, such as, for example, in a tablet-type personal computer or smart phone. In addition, computers such as computing system 52 may also include other peripheral output devices such as speakers 114 and printer 116, which may be connected through an output peripheral interface 118 or the like.

Computing system 52 may operate in a networked environment using logical connections to one or more remote computers, such as a remote computing system 120. The remote computing system 120 may be a personal computer (including, but not limited to, mobile electronic devices), a server, a router, a network PC, a peer device or other common network node, and typically includes many or all of the elements described above relative to computing system 52, although only a memory storage device 122 has been illustrated. The logical connections depicted include a local area network (LAN) 124 connecting through network interface 126 and a wide area network (WAN) 128 connecting via modem 130, but may also include other networks such as, for example, mobile telephone service networks. Such networking environments are commonplace in offices, enterprise-wide computer networks, intranets, mobile networks, and the Internet.

For example, in the present embodiment, computer system 52 may comprise the source machine from which data is being generated/transmitted and the remote computing system 120 may comprise the destination machine. Note however that source and destination machines need not be connected by a network or any other means, but instead, data may be transferred via any media capable of being written by the source platform and read by the destination platform or platforms.

In another example, in the present embodiment, remote computing system 120 may comprise the source machine from which data is being generated/transmitted and computer system 52 may comprise the destination machine.

In a further embodiment, in the present disclosure, computing system 52 may comprise both a source machine from which data is being generated/transmitted and a destination machine and remote computing system 120 may also comprise both a source machine from which data is being generated/transmitted and a destination machine.

Referring to FIG. 3, for the purposes of this disclosure, it will be appreciated that remote computer 120 may include any suitable term such as, but not limited to “device”, “processor based mobile device”, “mobile device”, “electronic device”, “processor based mobile electronic device”, “mobile electronic device”, “wireless electronic device”, or “location-capable wireless device,” including a smart phone or tablet computer.

The central processor operating pursuant to operating system software such as, but not limited to, Apple IOS®, Google Android® IBM OS/2®, Linux®, UNIX®, Microsoft Windows®, Apple Mac OSX®, and other commercially available operating systems provides functionality for the services provided by the present invention. The operating system or systems may reside at a central location or distributed locations (i.e., mirrored or standalone).

Software programs or modules instruct the operating systems to perform tasks such as, but not limited to, facilitating client requests, system maintenance, security, data storage, data backup, data mining, document/report generation, and algorithm generation. The provided functionality may be embodied directly in hardware, in a software module executed by a processor, or in any combination of the two.

Furthermore, software operations may be executed, in part or wholly, by one or more servers or a client's system, via hardware, software module or any combination of the two. A software module (program or executable) may reside in RAM memory, flash memory, ROM memory, EPROM memory, EEPROM memory, registers, hard disk, a removable disk, a CD-ROM, DVD, optical disk, or any other form of storage medium known in the art. An exemplary storage medium is coupled to the processor such that the processor can read information from, and write information to, the storage medium. In the alternative, the storage medium may be integral to the processor. The processor and the storage medium may also reside in an application specific integrated circuit (ASIC). The bus may be an optical or conventional bus operating pursuant to various protocols that are well known in the art.

FIG. 4 shows a process flow for the method and system of the present disclosure, including at a functional level, components that may be associated for performing the method and providing the system functions of the present disclosure. Specifically, system architecture 150 may be considered to begin with patient or athlete 152 who may be following an ACL prevention protocol. Patient 152 may be video recorded using a three-dimensional camera 154 for recording live video position information in the XYZ coordinate space. Live video position data 156 feeds from 3D camera 154 to pre-filtering functions 158. Thereafter, pre-filtered video position data 160 may be stored either in local storage or cloud-based storage 162.

An important aspect of video position data 160 includes the receipt and use of three (3) data streams from the camera. These data streams include (a) 3D vector information per joint location; (b) depth image information; and (c) a raw video stream.

From storage 162, data 164 may transfer to vector processing function 166 to generate vector processed data 168 for further adjustment and filtering at baseline adjustment and filtering function 170. Adjusted and filtered data 172 may then feed to biometrical algorithm function 174 of the presently disclosed subject matter. Adjusting or comparing biometrically information processed biomechanical algorithm function 174 may be combined with normative values 176 and analyzed and further processed to provide inputs to results dashboards 178, still images or live video information, 180, graphical comparison functions 182, and provide a summary of key problems at 184.

Referring again to FIG. 4, 3-D camera 154 captures a variety of physical therapy tests for each subject by tracking all joints of the body from the trunk to the toes for each test. Once this is done, vector processing module 166, baseline adjustment and filtering module 170, and biomechanical algorithm module 174 accurately and efficiently calculate the results for each test focusing on joint angles, quality of movement, and relationship of each joint to each other. There are set normative values 176 established for each joint for each test. If the subject falls outside of normative values 176, the specific joint is highlighted with a color scheme (green=good, yellow=fair, red=bad). The software allows reviewing all of the data and results in a table, graph, and 3-D video form allowing us to review with the subject, identify impairments and weaknesses and ultimately improve our ability to prevent injury.

For process 150, patient 152 is first put through a series of physical stress movements to assess the joints of the lower body. All movements are recorded using 3-D camera 154. The files saved within storage 162 after an examination include live video and all critical anatomical points of the lower body in 3-D space (x,y,z). Biomechanical analysis software 174 accurately and efficiently calculates the results for each test focusing on joint angles, quality of movement, and relationship of each joint to each other.

Normative values 176 are provided for each joint for each test, if the subject falls outside normative values 176, the specific joint is highlighted with a color scheme (green=good, yellow=fair, red=bad). The software allows the clinician to review all of the data and results in a table, graph, still image and 3-D video form. This enables/identifies impairments and weaknesses and ultimately improves ability to prevent injury.

Process 150 combines accurate joint tracking with clinician's guidelines to automate recognition of any joint during a series of movements that may be at risk. Once a physical therapy program is developed and followed the patient can be measured, assessed, accurately and repeatable to monitor progress and update the therapy program as needed.

A novel aspect of the present disclosure includes the use of imaging systems biomechanical analysis and for cooperating with analysis processes for ACL prevention and rehabilitation developed herein is novel and not found in the literature or patents. For the present uses, general three-dimensional (3-D) cameras and point tracking methods are available in the art. Examples of such 3-D cameras and point tracking methods appear in U.S. Pat. Nos. 7,974,443, 8,009,022, and 8,933,876. U.S. Pat. No. 7,974,443 entitled “Visual Target Tracking Using Model Fitting and Exemplar,” relates to a method of tracking a target and analyzing the observed depth image with a prior-trained collection of known poses. U.S. Pat. No. 8,009,022 ('022 patent) entitled “Systems and Methods for Immersive Interaction with Virtual Objects,” relates to a system to present the user with a 3-D virtual environment and parses depth camera data to correlate a user position with a position in the virtual environment. And, U.S. Pat. No. 8,933,876 entitled “Three Dimensional User Interface Session Control,” describes a non-tactile 3-D user interface for assessing 3-D session controls using depth perception and recording. Other such systems may be adapted and employed for the purposes of the presently disclosed subject matter.

Thus, computing system 52 of FIG. 3 may be configured to perform process 150 of FIG. 4 to provide the target tracking methods described herein. However, it should be understood that computing system 52 is provided as a non-limiting example of a device that may perform such target tracking. Other devices are within the scope of this disclosure.

Process 150 of FIG. 4 includes generating a report file containing a complete report of the athlete's bodily strength and flexibility that may be adjusted or tailored for the medical professional or patient. The report and the present method and system for its generation provide new insights in ACL injury prevention that properly address the seriousness and lengthy recovery time for ACL injuries by providing a reliable, objective, measured process and resulting data to more precisely track athlete/patient bodily strength and flexibility in a single instant, as well as over repeated measurements.

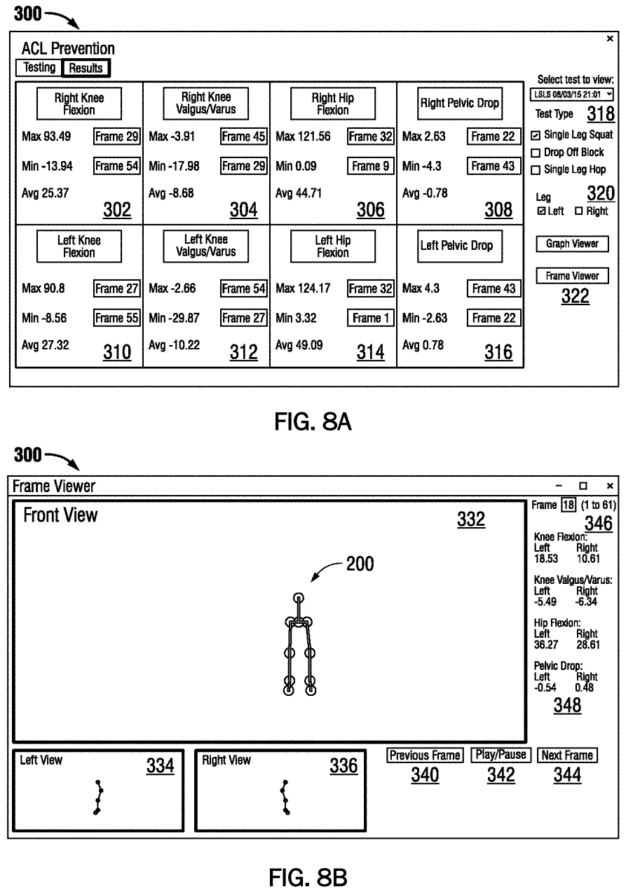

The present disclosure provides a robust and repeatable measure to guide the clinician and patient towards the correct strengthening and flexibly regimen. For instance, FIG. 8C, below, provides a results report for allowing a medical professional to also annotate notes to help clarify findings made possible through the subject matter of the present disclosure.

As a result of the processes for generating bodily strength and flexibility measurements, data presentation, and reporting, the present disclosure provides ways to accelerate ACL injury recovery. One appealing aspect of the present disclosure includes a means for accurately tracking and measuring all bodily joints through all possible movements. Using medical guidelines, all joint movements that fall outside of prescribed norms are quickly flagged on an easy to visualize dashboard. In addition, the present detailed and tailored report includes measured and accurate information relating to bodily strength and flexibility for progress tracking during a variety of therapy protocols.

Moreover, the disclosed subject matter provides the technical advantage of the ability to develop and understand trends among teams, positions, and players. That is, the present method and system provide both a clinical benefit and a benefit for coaches. For example, a coach who is recruiting two similar athletes may select one over the other simply due to a higher full body assessment score. Simply because due to superior flexibility/form/strength the risk of injury is lower. The counter could be used to make sure an athlete is at the same level or improved at the start of each season.

The presently disclosed method and system helps prevent ACL and related injuries, because analysis is measured and can be predictive, even prior to the onset of pain or inflammation. The present method and system also support improvements in sports performance, because of the ability measure and perceive improper joint movements, as well as to document such movements for general movement improvements while performing sports activities. Moreover, the present method and system may improve ways to perform orthopedic studies, document orthopedic experiments, as well as enrich orthopedic research literature with precise measurement that may advance related science and medical treatment.

A further advantage the present disclosure also includes the ability to assess conditions relating to other extremities, ligaments and tendons beyond the ACL. For example, upper extremities, such as elbow and shoulder ligaments may be measured objectively to assess conditions warranting attention.

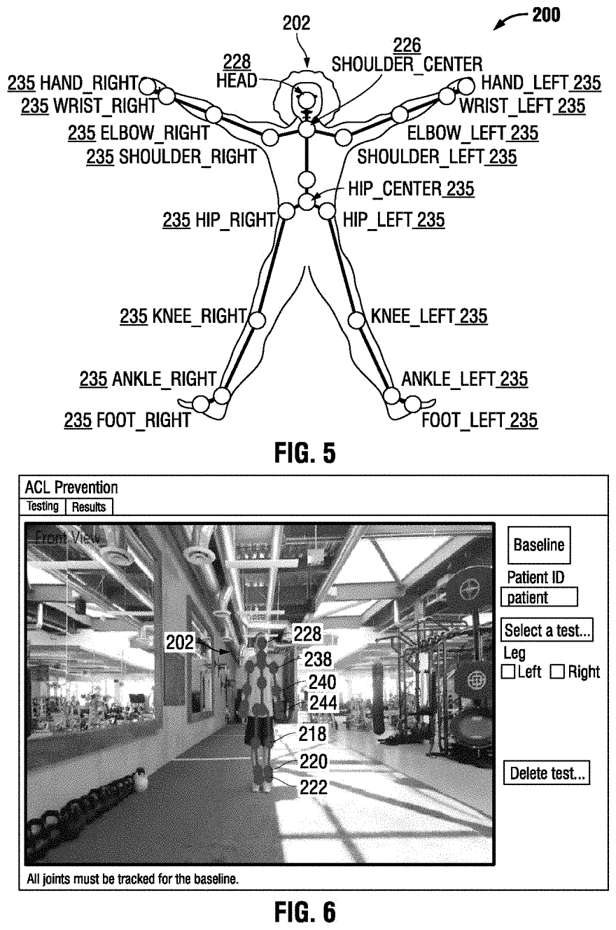

FIG. 5 illustrates the identification of selected data collection nodes for an exemplary subject applicable to the disclosed subject matter. For example, but not in a limiting way, FIG. 5 shows diagram 200 for showing human subject 202 for which frame of reference 204 may be established. In frame of reference 204, assigned nodes relating to human skeletal model 202 may include, for example, foot_right node 206, ankle_right node 208, knee_right node 210, hip_right node 212, hip_center node 214, hip_left node 216, knee_left node 218, ankle_left node 220, and foot_left node 222. Further measurements with human FIG. 202 may include spine node 224, shoulder_center node 226, and head node 228. Moreover, shoulder_right node 230, elbow_right node 232, wrist_right node 234, and hand_right node 326 may be measured using the node system of the present disclosure. This is true also for shoulder_left node 238, elbow_left node 240, wrist_left node 242, and hand_left node 244. With these nodes, the presently disclosed system provides a structured basis for determining the types of analyses of biomechanical functioning that may indicate the symptoms of ACL injury or other related problems.

FIG. 6 shows on computer user interface 250 a substantially frontal view of an exemplary skeletal model 202 overlaying a 3-D video image 252 of an athlete or subject. Thus, the process of the present disclosure biomechanically correlates and documents the association of 3-D video image 252 with skeletal model 202. Skeletal model 202 includes the exemplary skeletal nodes 206 through 244, as described above in FIG. 5.

In addition to showing exemplary skeletal model 202, FIG. 6 further shows on interface 250, control screen segment 256 for selecting baseline information, patient information, various tests, as well as sensing or collecting data on a right or left leg as well as “delete” and other management functions.

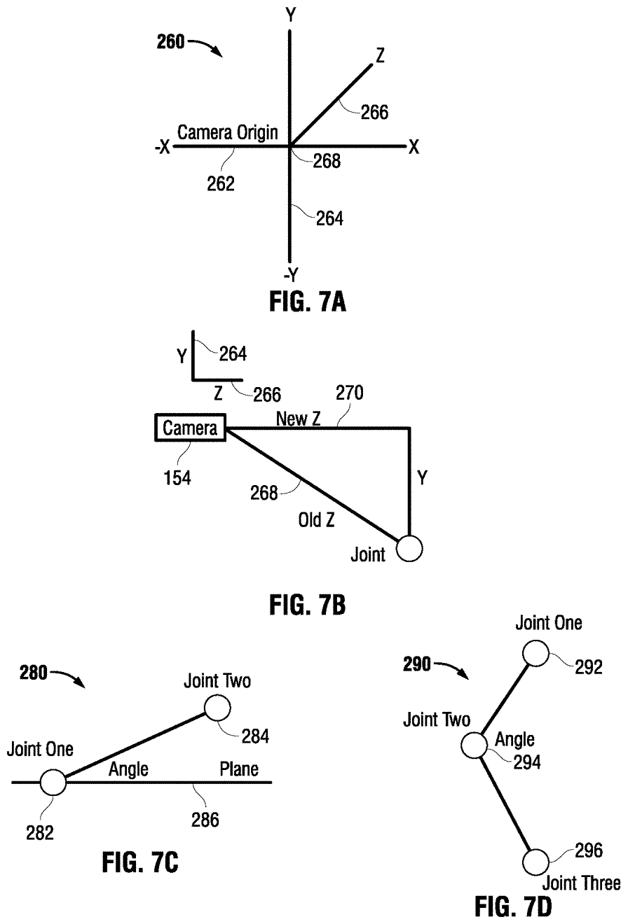

FIGS. 7A through 7D show various joint configurations for explaining the data capture and analysis for joint measurements according to the present teachings. In particular, FIG. 7A shows coordinate system 260, which includes X-axis 262, Y-axis 264, and Z-axis 266, which intersect to form camera origin 268. FIG. 7B illustrates how Y-axis 264 and Z-axis 266 may be sensed or determined by 3-D camera 154 for changing from one axis, old Z 268, to a new axis, new Z 270, as joint 272 changes along Z-axis 264.

With reference to computer user interface 250 of FIG. 6, the disclosed process and system employ 3-D sensing and extraction algorithms to capture firstly a baseline capture for modeling before running any tests are run a baseline is captured. The patient stands facing the camera in a neutral position, arms at side and feet pointed straight ahead and one frame of data is collected by the administrator from the baseline capture screen shown in FIG. 6. The baseline contains an X,Y,Z coordinate as shown in FIG. 7A, for each joint that is being tracked in that program. X and Y coordinates are taken directly from the camera and saved, but Z must be corrected. The Z coordinate the camera collects is an overall distance from the camera origin in all three coordinates, but the needed value is just the Z portion. To correct the X and Y distances are subtracted from the camera provided Z coordinate, shown in FIG. 7B. These coordinates will be used to make calculations that will be needed to make test specific calculations (e.g., pronation, supination).

From calculating a baseline, the present method and system allows for obtaining X, Y, and Z information against all the different movements captured by the 3-D camera 154. The generation of statistics both individually, as well as for a group of athletes/patients has great value. For example, in considering a football team, the present system would enable measuring and comparing the ranges for a given movement across the whole team. The generated statistics will have statistical outliers, a mean, and other data characteristics that might relate to strengths and/or weakness. Even an athletic trainer could use the measured and manipulatable data compare any given athlete to the team, and they can break it down even further analyses for generating training regimens or other purposes.

FIG. 7C shows 2-joint configuration 280 which includes joint 1 282 and joint 2 284, which are sensed relative to plane 286. And, FIG. 7D shows 3-joint configuration 290, wherein joint 292 connects to joint 2 294. Joint 2 294 connects to joint 3 296. Angle 298 separates joint 1 292 from joint 3 296, with which construction the disclosed process performs relevant analyses of join flexibility and stress.

The presently disclosed 3-D sensing and extraction algorithms include a baseline capture process. Before any tests are run a baseline is captured. The importance of the “Baseline Capture” and “Baseline Calculation” warrants special consideration. These are used to correct issues that found with the data we were receiving from the camera. Some were unexpected shifts of the joints, or calculating locations of joints of which the camera does not track. For each “Baseline Calculation” there is a description in the document that highlights the issue and how we fixed it.

Camera 154 calculates a Z coordinate as an overall distance from the camera origin. This means that the X and Y coordinates affect the Z coordinate; which is not necessary useful for purposes of the present calculations. Accordingly, the process corrects for this by subtracting the Z coordinate by the overall distance created by both the X and Y coordinates. The result becomes “new” Z coordinate which is only the distance in one coordinate system.

To illustrate the above concept, an athlete/patient stands facing the camera in a neutral position, arms at side and feet pointed straight ahead and one frame of data is collected by the administrator from the baseline capture screen shown in FIG. 7A. The baseline contains an X,Y,Z coordinate as shown in FIG. 7B, for each joint that is being tracked in that program. X and Y coordinates are taken directly from the camera and saved, but Z must be corrected. The Z coordinate that camera 154 collects is an overall distance from the camera origin in all three coordinates, but the needed value is just the Z portion. To correct the X and Y distances are subtracted from the camera provided Z coordinate, shown in FIG. 7C. These coordinates will be used to make calculations that will be needed to make test specific calculations (e.g., pronation, supination).

The raw coordinate data from a baseline capture are used as inputs to our algorithms that correct for nonlinearities in the 3-D camera. This allows the method and system, for example, to create pattern match filters, apply min/max windowing to threshold the data, and nonlinear correction algorithms. This baseline can be used to optimize future algorithms as part of the raw data processing.

The present process further includes Y reference base calculations, as herein explained. For forward facing angles where X and Y coordinates are required, the Y coordinate of a joint must be autocorrected applying our algorithm and the baseline. For example, when a patient squats down the 3-D vectors sensed between points have a nonlinear angular skew for angles utilizing the x coordinates between points. This issue is a correctable property of the sensor. Utilizing the baseline and applying our algorithm we accurately correct for this nonlinear skew. The joints that need this baseline calculation are the right hip, left hip, right knee, left knee, right ankle, and left ankle.

These Y reference calculations are equal to the baseline Y position of the particular joint.
rightHipYRef=Right Hip Y
leftHipYRef=Left Hip Y
rightKneeYRef=Right Knee Y
leftKneeYRef=Left Knee Y
rightAnkleYRef=Right Ankle Y
leftAnkleYRef=Left Ankle Y

The present system further performs hip reference distance calculations. When a patient squats down the 3-D vectors sensed between points have a nonlinear skew that effects X coordinates for the hips as the Y coordinate gets closer to the Y coordinate for the knees. Baseline values are used for the distance between each hip and the mid spine to correct the hip x coordinate. This algorithm in effect applies a nonlinear correction to maintain a constant hip to knee distance.

These are calculated by getting the distance between the mid spine and hip joints in X, Y, and Z planes according to the following equations:

$$rightHipRefDistance = \sqrt{\begin{matrix}\begin{matrix}{( BaseSpineX - RightHipX )}^{2} + \\ {( BaseSpineY - RightHipY )}^{2} +\end{matrix} \\ {( BaseSpineZ - RightHipZ )}^{2}\end{matrix}}$$

$$leftHipRefDistance = \sqrt{\begin{matrix}\begin{matrix}{( BaseSpineX - LeftHipX )}^{2} + \\ {( BaseSpineY - LeftHipY )}^{2} +\end{matrix} \\ {( BaseSpineZ - LeftHipZ )}^{2}\end{matrix}}$$

The present system employs a heal point algorithm, in addition to the standard joints tracked. This novel algorithm has use for tracking new locations on the body driven by physiological tracking requirements for Full Body Assessment (FBA) for example. Using the X and Z measurements for the ankle and foot the associated heal point is calculated using the following equations:

$$rightFootZRef = \sqrt{\begin{matrix}{( Right Ankle X - Right Foot X )}^{2} + \\ {( Right Ankle Z - Right Foot Z )}^{2}\end{matrix}}$$

$$leftFootZRef = \sqrt{\begin{matrix}{( Lef t Ankle X - Lef t Foot X )}^{2} + \\ {( Lef t Ankle Z - Lef t Foot Z )}^{2}\end{matrix}}$$

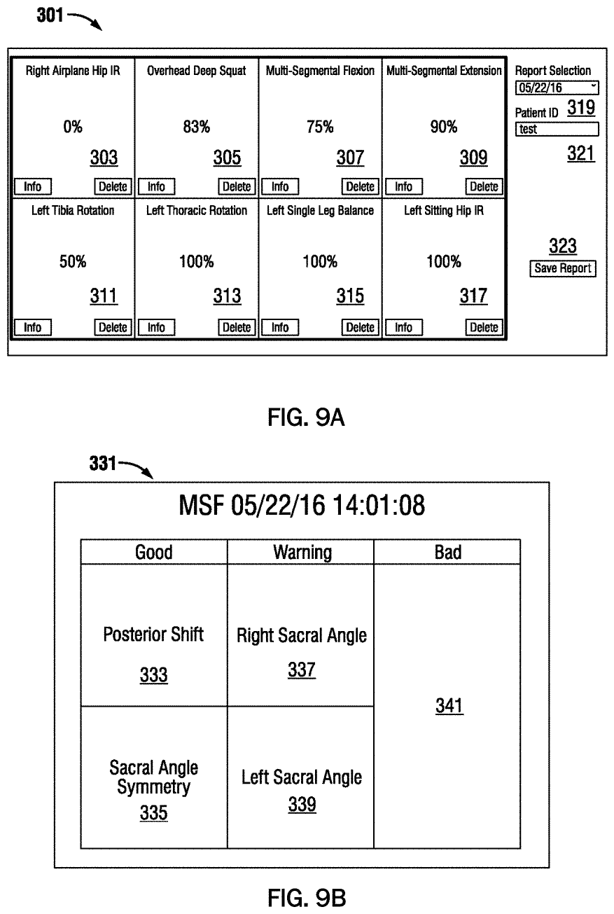

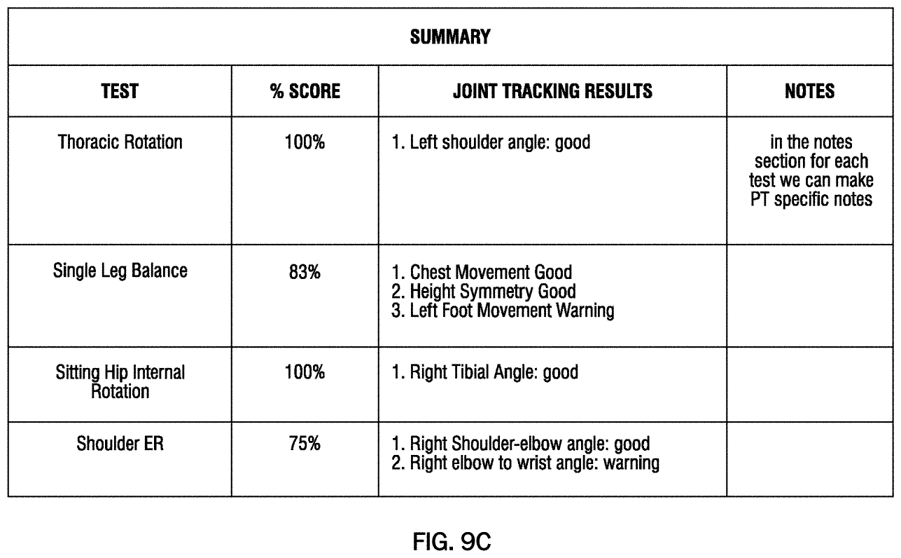

The testing capture processes are as follows. The administrator starts capturing from the testing capture screen (see FIG. 8) and then has the patient perform the specified test. For each frame of the test an X,Y,Z coordinate is collected for every joint, with the Z correction also applied (See FIG. 9). The raw data from the tests is stored and not analyzed until it is reviewed.

Following the above measurements, the method and system enable post processing. The stored data from the test capture is handled by the software to relay pertinent information. First the raw position data is filtered to remove any anomalies captured by the camera. Depending on the test, there are certain calculations needed to properly assess the patient's performance. Each algorithm is run to calculate the results and then are displayed on the results screen shown in FIG. 5.

The present method and system perform angle calculations for generating data relevant to bodily strength and flexibility. An angle calculation uses an algorithm to calculate the angle created by two or three joints in any two planes needed depending on the needed angle. Angles formed by looking straight on the patient use the X and Y planes, while angles formed by looking from the side of the patient use Y and Z.

A first angle calculation of the present embodiment may be a two-joint angle calculation. An angle that uses two joints is formed by a line between the two joints and a flat plane in any of the three axes as shown in FIG. 6. An angle between two joints is calculated by taking a difference of the joints for each plane then taking the inverse tangent of the result of dividing the difference in the axis the plane is not in divided by the difference in the axis the plane is in. The value is then multiplied by 180/[Equation] to convert to degrees. A positive or negative is assigned to the angle depending on if the first joint has a greater or smaller value in the comparative axis. The same angles can have different names depending on where joint one is in relation to two, so the positive or negative is used to differentiate between them (eg. Pelvic drop, trunk lean).

The present system enables the automated calculation of a pelvic drop/trunk lean. This angle is created by both the right and left hip X and Y coordinates and a plane in the Y coordinates. When the hip this angle is being measured on is above the other it is called trunk lean and when it is below the other it is called pelvic drop.

difX=|hipOne·X−hipTwo·X|

difY=|hipOne·Y−hipTwo·Y|

positiveOrNegative=1 if hipOne·Y>hipTwo·Y=−1 if hipOne·Y<hipTwo·Y

angle=(tan⁻¹(difY÷difX)×180÷π)×positiveOrNegative

Another important calculation of the present embodiment includes a three joint angle calculations. Angles formed between three joints are calculated by creating two separate vectors, one between joint one and joint two, and the second between joint two and joint three (see FIG. 7). Then the lengths of the vectors are calculated in the required only the two required axes in order to be used to normalize both vectors. The dot product is then taken between the two vectors and the inverse cosine is taken of the dot product to get the angle in radians. The value is then multiplied by 180/[Equation] to convert to degrees. A positive or negative value is assigned to the angle depending where joint two is compared to joint one and three. The same angles can have different names depending on which way they are pointing so this positive or negative is used to differentiate between them (e.g., valgus, varus).

The present embodiment further calculates a hip flexion measurement and generates report data relating to these calculations. This angle is created by the mid spine, hip, and knee, of the same leg, in the Y and Z coordinates, with the angle occurring at the knee.

vectorOne=Vector between Knee and Hip={Y=Knee Y−Hip Y,Z=Knee Z−Hip Z}

vectorTwo=Vector between Hip and Mid Spine={Y=Mid Spine−Hip Y,Z=Mid Spine Z−Hip Z}

vectorOneLength=\sqrt{vectorOne·Y²+vectorOne·Z²}

vectorTwoLength=\sqrt{vectorTwo·Y²+vectorTwo·Z²}

positiveOrNegative=Sign((Knee Z−Mid Spine Z)×(Hip Y−Mid Spine Y)−(Knee Y−Mid Spine Y)×(Hip Z−Mid Spine Z))
Either a 1 or −1 used to determine if angle is a flexion or hyperextension angle

Normalize vectorOne={Y=vectorOne·Y÷vectorOneLength,Z=vectorOne·Z÷vectorOneLength}

Normalize vectorTwo={Y=vectorTwo·Y÷vectorTwoLength,Z=vectorTwo·Z÷vectorTwoLength}

dotProduct=vectorOne·Y·vectorTwo·Y+vectorOne·Z×vectorTwo·Z

angle=(cos⁻¹(dotProduct×180÷π)

For knee flexion measurements, the present embodiment performs automatically the calculations explained here. The angle is created by the hip, knee, and ankle, of the same leg, in the Y and Z coordinates, with the angle occurring at the knee.

vectorOne=Vector between Hip and Knee={Y=Hip Y−Knee Y,Z=Hip Z−Knee Z}

vectorTwo=Vector between Hip and Mid Spine={Y=Ankle Y−Knee Y,Z=Ankle Z−Knee Z}

vectorOneLength=\sqrt{vectorOne·Y²+vectorOne·Z²}

vectorTwoLength=\sqrt{vectorTwo·Y²+vectorTwo·Z²}

positiveOrNegative=Sign((Hip Z−Ankle Z)×(Knee Y−Ankle Y)−(Hip Y−Ankle Y)×(Knee Z−Ankle Z))
Either a 1 or −1 used to determine if angle is a flexion or hyperextension angle

Normalize vectorOne={Y=vectorOne·Y÷vectorOneLength,Z=vectorOne·Z÷vectorOneLength}

Normalize vectorTwo={Y=vectorTwo·Y÷vectorTwoLength,Z=vectorTwo·Z÷vectorTwoLength}

dotProduct=vectorOne·Y×vectorTwo·Y+vectorOne·Z×vectorTwo·Z

angle=(cos⁻¹(dotProduct×180÷π)

The present embodiment further enables automated knee valgus/varus measurements and calculations. This angle is created by the hip, knee, and ankle of the same leg, in the X and Y coordinates, with the angle occurring at the knee. For this calculation the hipRefDistance baseline value needs to be used to correct the hip x position that was captured during the test that is thrown off when the patient bends at the knees.

$$hipXPosition = Base Spine X - \sqrt{\begin{matrix}\begin{matrix}{hipRefDistance}^{2} - \\ {( Base Spine Y - Hip Y )}^{2} -\end{matrix} \\ {( Base Spine Z - Hip Z )}^{2}\end{matrix}}$$

$$positiveOrNegative = Sign ( ( hipXPosition - Ankle X ) \times ( Knee Y - Ankle Y ) - ( Hip Y - Ankle Y ) \times ( Knee X - Ankle X ) )$$

$$vectorOne = \{ X = hipXPosition - Knee X , Y = hipYRef - kneeYRef \}$$

$$vectorTwo = \{ X = Ankle X - Knee X , Y = ankleYRef - kneeYRef \}$$

$$vectorOneLength = \sqrt{vectorOne \cdot {X}^{2} + vectorOne \cdot {Y}^{2}}$$

$$vectorTwoLength = \sqrt{vectorTwo \cdot {X}^{2} + vectorTwo \cdot {Y}^{2}}$$

$$Normalize vectorOne = \{ X = vectorOne \cdot X \div vectorOneLength , Y = vectorOne \cdot Y \div vectorOneLength \}$$

$$Normalize vectorTwo = \{ X = vectorTwo \cdot X \div vectorTwoLength , Y = vectorTwo \div vectorTwoLength \}$$

$$dotProduct = vectorOne \cdot X \times vectorTwo \cdot X + vectorOne \cdot Y \times vectorTwo \cdot Y$$

$$angle = ( 180 - ( {cos}^{- 1} ( dotProduct \times 180 \div π ) ) \times positiveOrNegative$$

This angle is created by the knee, ankle, and foot of the same leg, in the X and Y coordinates, with the angle occurring at the ankle. When calculating the footDistance baseline value needs to be used to calculate the footXPosition which represents the x value of the heel.

footXPosition=Foot X+ or −\sqrt{footDistance²−(Ankle Z−Foot Z)²}

positiveOrNegative=Sign((Knee X−footXPosition)×(Ankle Y−Foot Y)−(Knee Y−Foot Y)−(Ankle X−Foot X))

vectorOne={X=Knee X−Ankle X,Y=Knee Y−Ankle Y}

vectorTwo={X=footXPosition−Ankle X,Y=Foot Y−Ankle Y}

vectorOneLength=\sqrt{vectorOne·X²+vectorOne·Y²}

vectorTwoLength=\sqrt{vectorTwo·X²+vectorTwo·Y²}

Normalize vectorOne={X=vectorOne·X÷vectorOneLength,Y=vectorOne·Y÷vectorOneLength}

Normalize vectorTwo={X=vectorTwo·X÷vectorTwoLength,Y=vectorTwo÷vectorTwoLength}

dotProduct=vectorOne·X×vectorTwo·X+vectorOne·Y×vectorTwo·Y

angle=(180−(cos⁻¹(dotProduct×180÷π))×positiveOrNegative

FIG. 8A shows an image of the collected data points for a front view of an individual for assessing ACL related information. FIG. 8A provides the image viewer with the background removed to show exemplary skeletal formation 200 according to the teachings of the presently disclosed subject matter. In such image, in addition to front view. Frame viewer 330 includes front view 332, left view 334, right view 336, and control screen segment 338. For each screen segment, previous frame select 340, play/pause select 342, and next frame select 344 appear. In screen segment 346, data relating to knee flexion information, left and right, appear. For example, a left knee flexion in the example of FIG. 8B shows a flexion value of 18.53 for the left and 10.61 for the right. In addition, similar data, i.e, left knee valgus/varus value of −5.69 and right of −6.34 are measured. Furthermore, hip flexion left at 36.27 and right at 28.61 and pelvic drop for left is −0.88 and left, 0.88 appear. This information is precise, recorded, and provides data points that are relevant to the physiological characteristics of the subject.

The presently disclosed method and system has the ability to automatically perform the above calculations to generate, record, analyze, and report measurement, such as the above, to determining a patient's or athlete's bodily strength and flexibility. The results of the described process are here explained further. FIG. 8A portrays a computer screen interface 300 for reporting physiological data derived from the 3-D camera recordings and the processes of the present disclosure and relating ACL injury prevention. In screen 300 appears data relating to right knee flexion 302, right knee valgus/varus 304, right hip flexion 306, right pelvic drop 308, left knee flexion 310, left knee valgus/varus 312, left hip flexion 314, and left pelvic drop 316. Also, control screen segment 318 permits the selection of which test to view, as well as selection of a particular test type in screen segment 320 such as single leg squat, drop off block, or single leg hop. Screen segment 320 further permits selection between displaying data on a left or right leg, determining a graph viewer to go to a graph, or to a frame viewer to show a video image.

FIG. 9A presents report 301 for displaying results of the measurements of the presently disclosed subject matter. Particularly, report 301 includes Right Airplane Hip result 303 showing a 0% value in red to indicate a problem measurement, an Overhead Deep Squat result 305 in green to indicate a good measurement of 87%, a Multi-Segmental Flexion result 307 showing a good measurement of 75% in green, and a good measurement of 90% in green for Multi-Segmental Extension result 309. Moreover, a potentially troublesome measurement of 50% in yellow for Left Tibia Rotation result 311, and good results in green numerals for Left Thoriac Rotation result 313, Left Single Leg Balance result 315, and Left Sitting Hip IR result 317, all measuring 100%. Report 301 further shows Report Selection field 319, Patient ID field 312 and Save Report control icon 323 for controlling whether the user may save the report for further use.

FIG. 9B presents report 331 for displaying results of the measurements of the presently disclosed subject matter for showing measurements that have Good, Warning, and Bad indications. In the example of FIG. 9B, report 331 for subject MSF, taken on May 22, 2016 at 14:01:08 reveals that measurements for Posterior Shift result 333 and Sacral Angle Symmetry result 335 as Good, measurements for Right Sacral Angel result 337 and Left Sacral Angle 339 at Warning indications, and no results for Bad measurement 341.

FIG. 9C presents an exemplary report made possible by the teachings of the present disclosure that includes a summary for each test, including a % score, joint tracking results, and relevant notes. For the example of a Thoracic Rotation test, a 100% score was measured in which a left shoulder angle was measure to be good. As FIG. 9C indicates, in the notes section for each test the present method and system can make PT specific notes. Also for a single leg balance, a 83% score was recorded wherein (1) Chest Movement was measured “Good”; (2) Height Symmetry was “Good,” and (3) Left Foot Movement measured in a Warning, and not Good state. Finally, FIG. 8C shows that for a sitting hip internal rotation test, a 100% score was measured wherein the right tibial angle measured good. And, lastly for the Shoulder ER test, a measured score was 75% with the right shoulder-elbow angle determined to be good. Here the right elbow to wrist angle measured in a warning state. Thus, the summary report of FIG. 8C provides a summary of recommendations for use by the patient, client and/or athlete.

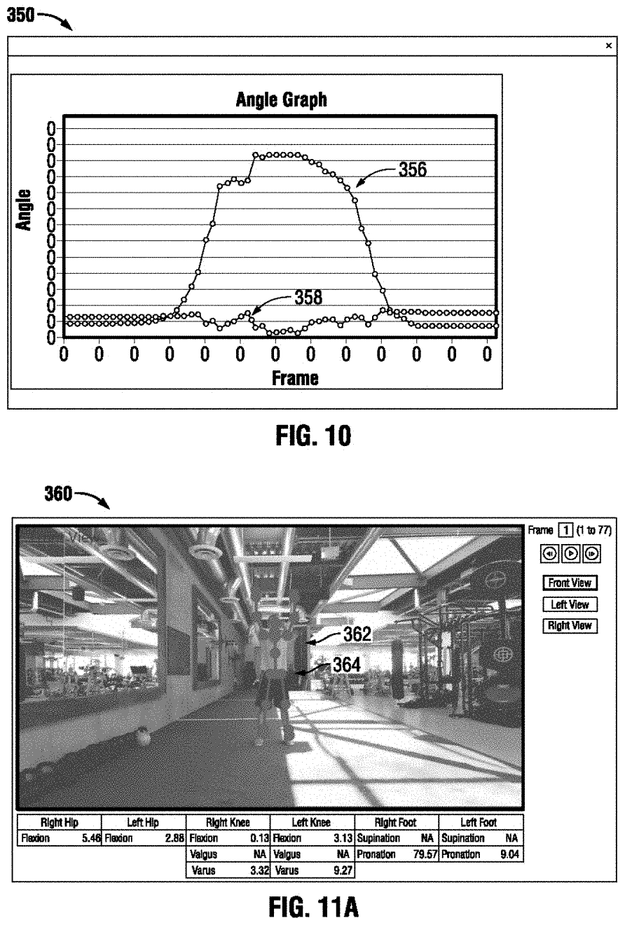

FIG. 10 presents graphs of collected data points relating to right knee measurements of an individual. FIG. 10 shows graph viewer screen 350 of the presently disclosed subject matter. In screen segment may be controls for generating graphs relating to the data sensed and recorded and reported in the 3-D data collection for ACL measurement display 300 of FIG. 8A and frame viewer display 301 of FIG. 9A. In particular, angle graph 354 of graph viewer screen 350 shows as graph 356 the results of selecting right knee flexion in screen segment 352. Graph 358 shows the result of selecting right knee valgus/varus data in screen segment 352. With the information here disclosed, it is now possible to begin the analysis of data for a given subject as a set of motions, such as a sports-related motion might occur.

In part, because of the measurement, reporting, and tracking aspects of the present disclosure, a clinician may have a patient run through a wide array of tests for following bodily strength and flexibility examination program. The present method and system enable calculating measurements that will highlight the patient's weaknesses and strengths in regards to what is being tested for the particular program. As here shown, the disclosed subject matter enables generating a report containing a grade for each test and a breakdown of how the patient did for each measurement (i.e., Good, Warning, Bad). Based on the report, the clinician may assess the patient's performance and assign an appropriate regiment to improve performance or decrease risk for injury, such as an ACL injury. After the regiment is completed, the clinician may have the patient rerun tests for the program for comparing the new report the original other secondary measurements to show how the patient has progressed.

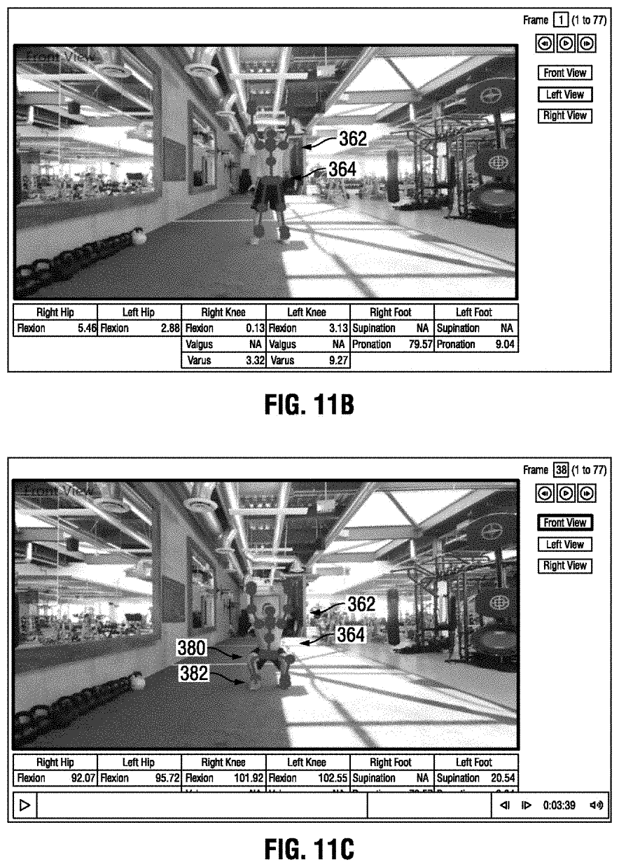

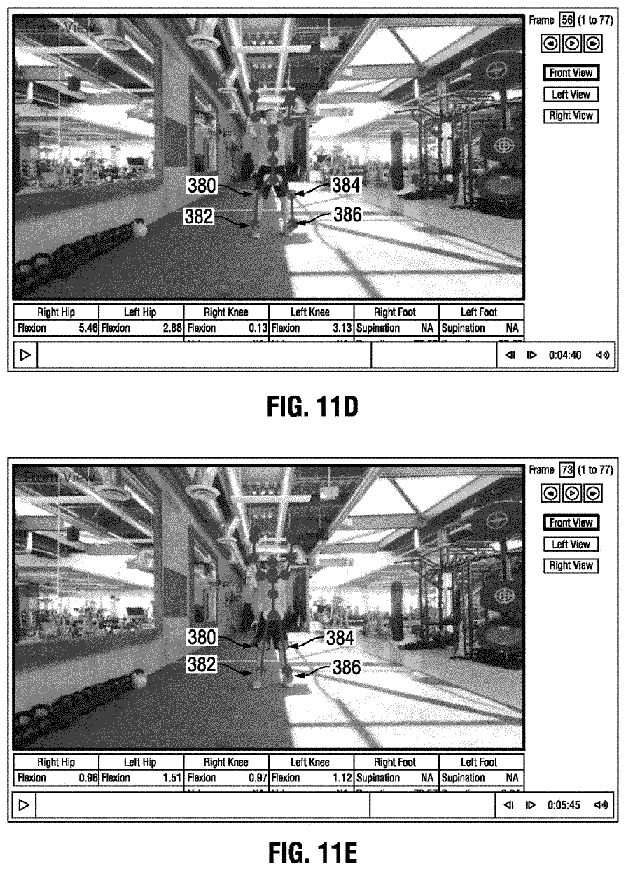

FIGS. 11A through 11E appear a frame viewer screen 360 showing a 3-D video of subject 362 during a double-leg squat motion test of flexion, varus, and pronation employing the method and system of the present disclosure. The present disclosure generates a three-dimensional videotape of subject 362 associated with skeletal framework 364 for generating data in table 366, according to the menu functions of screen sub-portion 368. As subject 362 performs a double-leg squat, data generated for right hip flexion 368 and left hip flexion 370, as well as right knee flexion and varus 372, left knee flexion and varus 374, and right foot pronation 376 and left foot pronation 378 are recorded and displayed in frame viewer screen 360.

FIG. 11B continues to demonstrate how the skeletal pattern 364 tracks the movement of subject 362 and generates varying data for the various right hip, left hip, and other data values, flexion, et cetera, for the subject. With particular attention to FIG. 11C, when subject (362) moves to create a symptom or indication of a condition prone to cause an ACL injury or sensitivity, the color of the associated nodes 380 and 382 changes to indicate a potential problem. FIGS. 11D and 11E further illustrate how the information relating to right and left knee and right and left foot are creating ACL inducing values. The data display can help to isolate and determine that the subject 362 motions are likely to be productive of an ACL injury condition. FIG. 11E further displays how in the completion of the double-leg squat show the indications of ACL affecting motions through the displayed red nodes 384 and 386.

Thus, the sequence of 3-D videos of FIGS. 11A through 11E depict how ACL prevention program may be performed through the drop down squat test. The clinician first positions a patient the correct distance from the 3-D camera and captures a baseline image. The baseline is captured as the person is standing comfortably upright. All anatomical locations for the lower body are captured in 3-D space (X,Y,Z). After the baseline image the drop down squat test is selected. The patient is positioned on top of a platform n-inch off of the ground and asked to jump down in a controlled fashion into a squat position and then return to standing.

The results from the most recent software analysis testing included five subjects. The subjects were each ran through all of the ACL prevention tests and the results for each individual from the testing are summarized below:

Subject One:

Overall this subject demonstrated a lot of instability and poor motor control in his knees and ankles. For all of the tests this was dictated by the software and 3-D camera with yellow and red dots over these joints and for the results section both in graph and table form, demonstrated joint angles outside of the well-established normative values. The dynamic tests such as triple hop for distance, T test, and acceleration/deceleration demonstrated the instability and lack of motor control the most for this subject. Both the knee and ankle joint movements for each test were validated by both the video analysis software and the trained visual eye of doctor of physical therapists.

Subject Two:

The subject primarily demonstrated the improper shock absorption through his hips and knees with the drop down squat test, single leg hop test, and triple hop test. The subject demonstrated an upright posture during all of these tests causing him to fall outside the norms established for both the hips and knees for each test. This was demonstrated by the 3-D camera highlighting the hips and knees with red and yellow dotes in the testing and results section, the graph and table for this subject. Both the hip and knee joint movements for each test were validated by both the video analysis software and the trained visual eye of doctor of physical therapists.

Subject Three:

The subject demonstrated significant weakness, poor motor control and balance with the ACL prevention tests with the source of majority of these impairments being at the hip and knee joints. For majority of the tests, the subject fell outside the norms for pelvic drop and knee valgus especially with single leg squat, single leg hop, and triple hop tests. Both the hip and knee joint movements for each test were validated by both the video analysis software and the trained visual eye of doctor of physical therapists.

Subjects Four and Five:

Both subjects four and five demonstrated proper form for all tests and fell within the normative values established for each joint in the software. This was demonstrated in the results section with video, graphs, and tables. All joint movements for each test were validated by both the video analysis software and the trained visual eye of doctor of physical therapists.

The invention can be used to provide a more accurate measurement of joint angles as they are moved through an array of ACL prevention movements (single leg squat, box jump-squat, T-test . . . ). The improved repeatable measurement accuracy, time saving efficient use model, and the ability to track and measure long term results over populations and/or specific patients summarize the key benefits.

The invention can be extended for accurate measurement and assessment of any joint of the body (e.g. lower back, shoulder, elbow, etc.). The consistent repeatable accuracy and digital storage allows for exploration of cause and effect studies and associated scientific advancements. For example if ‘n’ football teams are screened at the start of a season. At the end of the season all ACL injured players can then be compared for like weaknesses. This can will lead to improved strengthening and flexibility protocols.

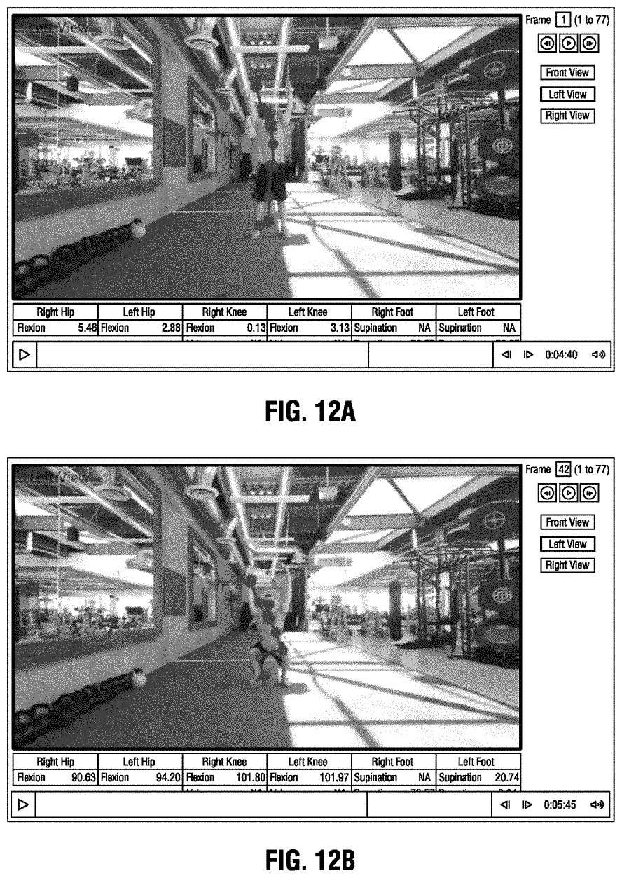

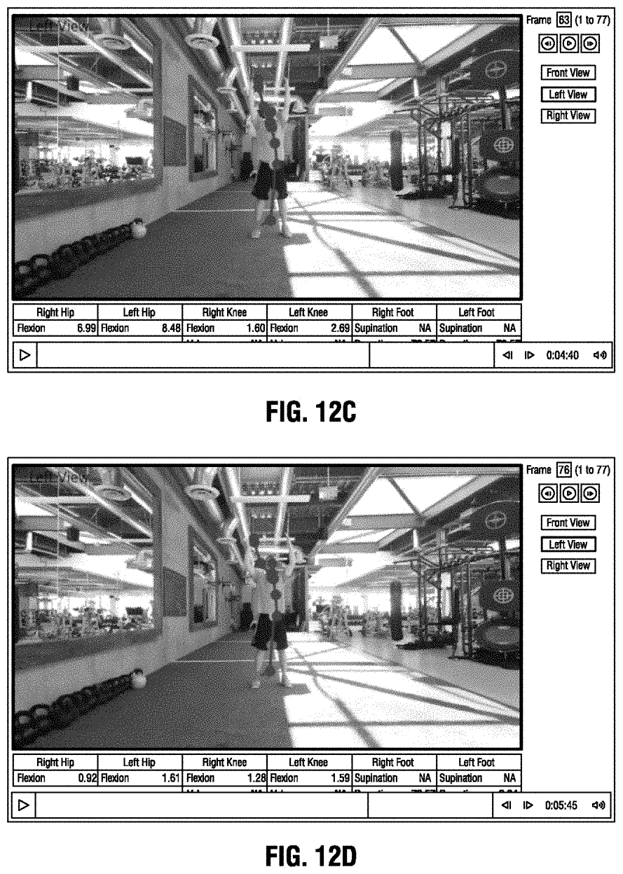

FIGS. 12A through 12D present a side display of data collection nodes overlaying a front image of an athlete for analyzing a double leg squat test according the present teachings. FIGS. 12A through 12D illustrates a further aspect of the present disclosure. In particular, the presently disclosed system provides for side views of subject 362 by demonstrating a side skeletal aspect 390, as subject 362 performs a double-leg squat. Note that the skeletal model 390 is actually a side view, whereas the video image of subject 361 appears in this instance as a front view. With particular attention to FIGS. 12B through 12D there appears in skeletal formation 390, red nodes 392 and 394 depicting measurements suggestive of ACL damaging positions.

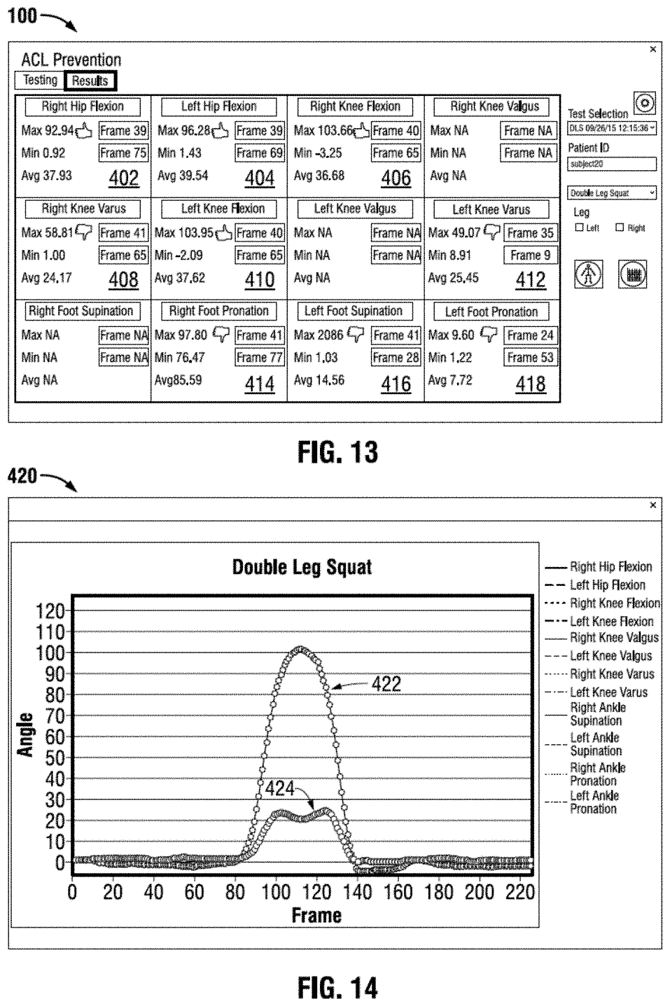

FIG. 13 shows an ACL prevention data dashboard for indicating whether the method and system of the present disclosure has determined an ACL injury likely condition. FIGS. 13 and 14 demonstrate the effect of the measurements taken with regard to ACL prevention and as displayed in FIGS. 11A through 12D associating with the double-leg squat maneuver. Thus, right flexion data screen portion ACL results screen 400 shows right hip flexion screen portion 402 with a green thumbs-up indicator showing there not to be an ACL injury inducing measurement. The same is true for left hip flexion screen portion 404 and right knee flexion portion 406. In this instance, right knee valgus data is not shown whereas right knee varus data 408 shows a damaging or limited measurement for right knee varus, as shown by the red thumbs-down indicator. In each of the sub-screens appears the identifying frame where such information appears. There is a maximum value and a minimum value for each of the parameters and the frame identifies where such value may be seen. Continuing in screen 400 left knee flexion measurement 410 shows a good indication, whereas right foot pronation 414, left foot supination 416, and left foot pronation 418, as well as left knee varus 412 data all indicate an ACL damaging motion.

FIG. 14 shows in graph viewer screen 420 results including right knee flexion data on graph 422, as well as right knee valgus and right knee varus data on graph 424. Graphs indicative of the data collected associated with these measurements. All for the double-leg squat.

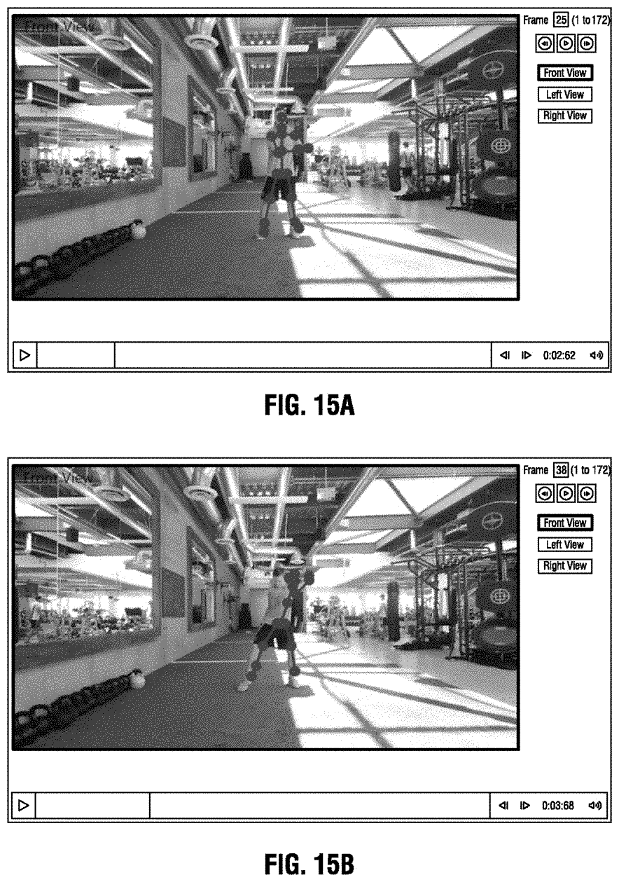

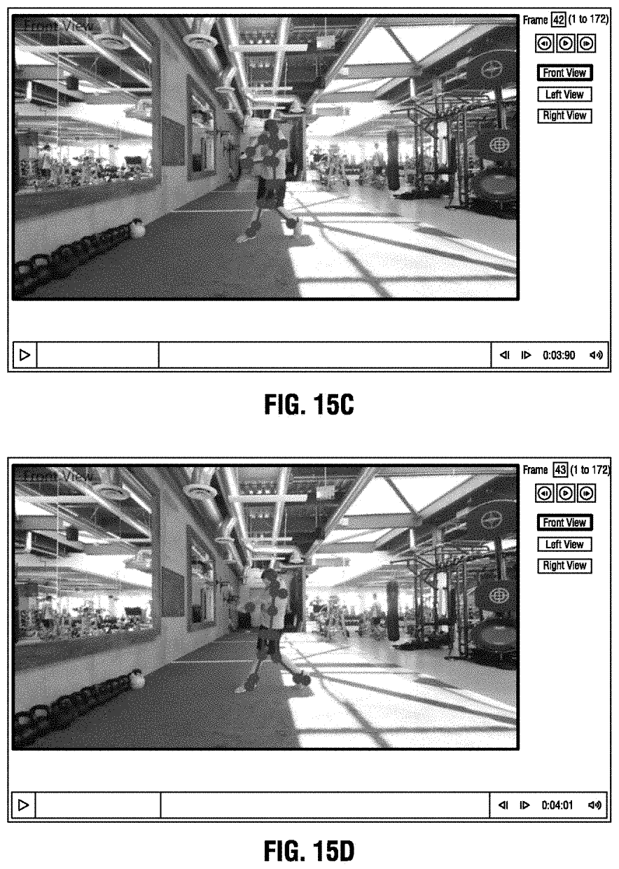

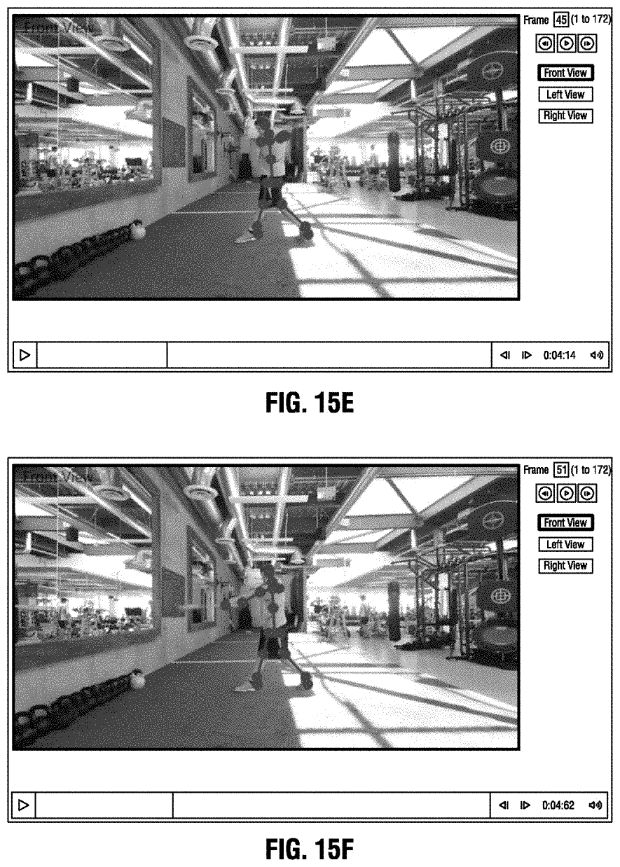

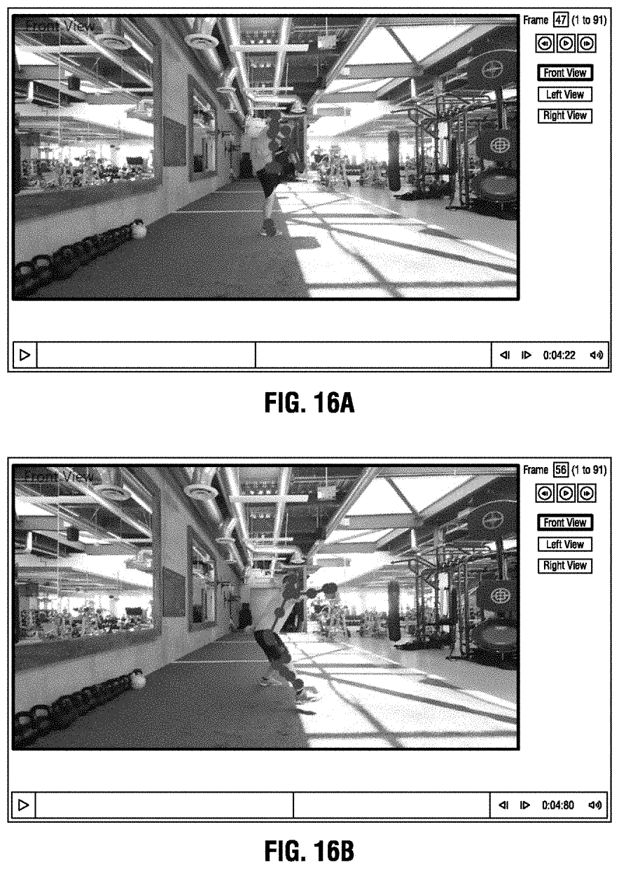

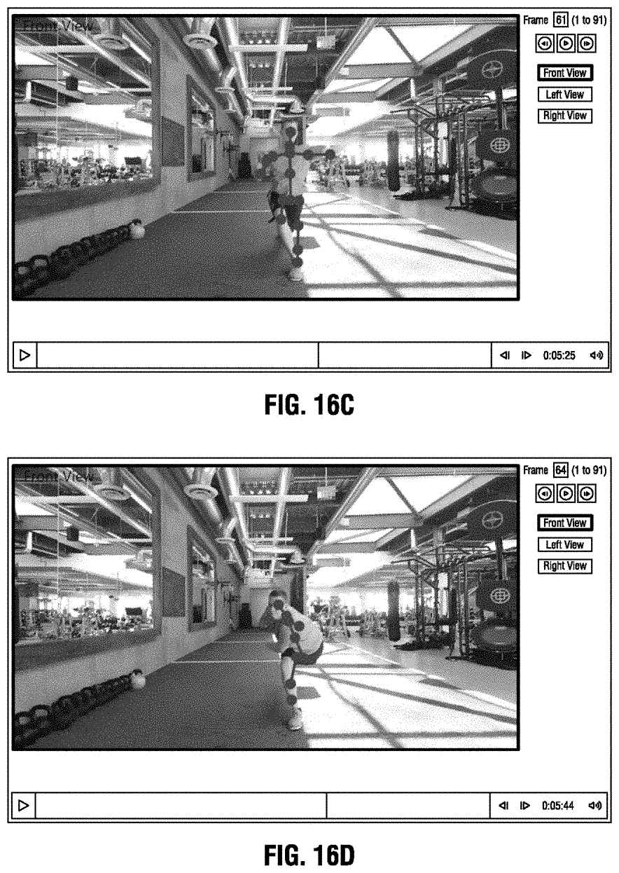

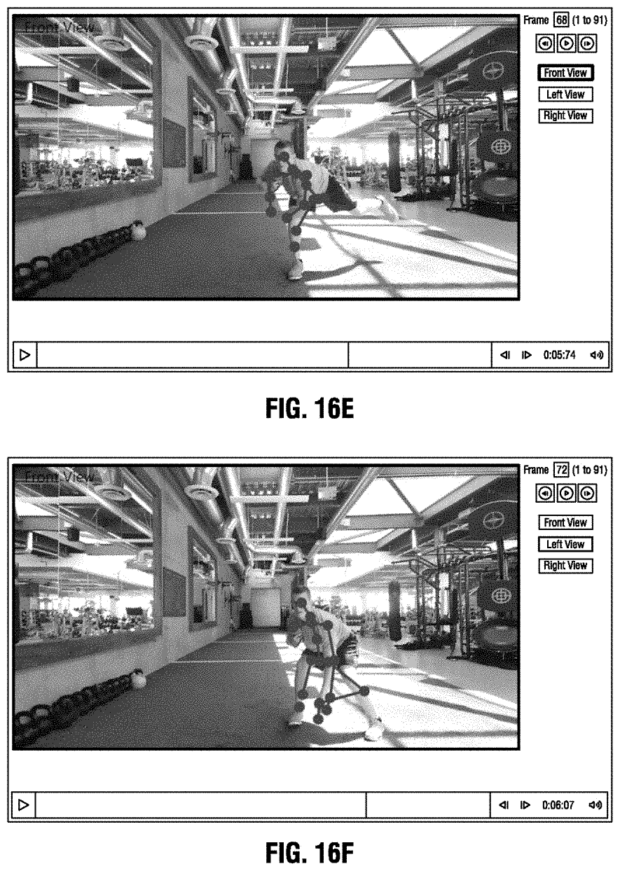

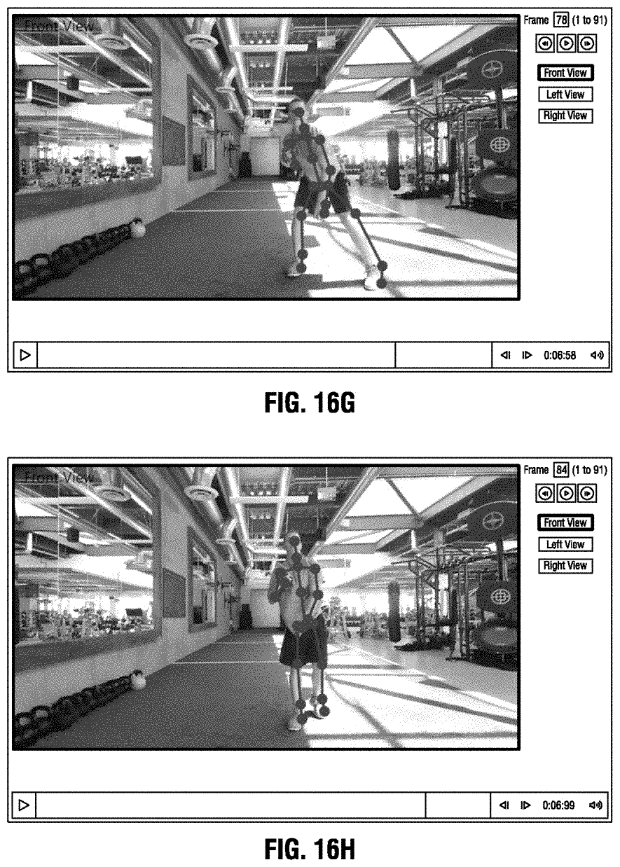

The method and system have utility beyond the determination of ACL injury inducing motions by a subject. For example, FIGS. 15A through 15F illustrate how the system of a present disclosure may be used to diagnose or to analyze particular sporting operations such as the swing of a bat by a baseball player, or in FIGS. 16A through 16H the pitching motions of a baseball pitcher. As FIGS. 15A through 15F, and FIGS. 16A through 16H show, the system and method here disclosed also track points that are an extension of the body, such as a bat. For example the (X,Y,Z) coordinates of the end of the bat allow for the calculation of bat speed and trajectory as a person swings down and follows through. These disclosures, this information, and it's use for analyzing the effectiveness of an athlete and sensing conditions where such sports operations can be analyzed, diagnosed, and improved provide yet another application of the method and system of the present disclosure.

In light of the above, the present disclosure provides, a method and system for automated biomechanical analysis that enables, as an illustrative example, anterior cruciate ligament (ACL) injury prevention and recovery. The method and system provide for processing an automated ACL injury analysis algorithm on a computer processor. The method and system further provides for recording measurements relating to a plurality of physical therapy tests of a subject using a three-dimensional measurement imaging device associated with said computer processor. By recording such measurements, the present disclosure allows for tracking the movement of joints on the subject's body from the subject's trunk downward to the subject's toes for each of said plurality of physical therapy tests using the associated imaging device and computer processor. These recorded measurements are then available for the computer processor to calculate the results of said plurality of physical therapy tests using said automated ACL injury analysis algorithm. The ACL injury analysis algorithm provides instructions for the computer processor to analyze angles, movement quality, and related interrelationships amongst said joints. The disclosed subject matter further enables the computer processor to derive comparisons of the calculated results with a plurality of normative values stored on the computer process. The computer processor further may associate indicators for said comparisons. The computer process stores or otherwise accesses the associated indicators and relates the indicators to the potential of the subject for experiencing an ACL injury. These indictors and the potential for ACL injury are further made available on one or more displays associated with the computer processor.

All references, including publications, patent applications, and patents, cited herein are hereby incorporated by reference to the same extent as if each reference were individually and specifically indicated to be incorporated by reference and were set forth in its entirety herein.

The methods, systems, process flows and logic of disclosed subject matter associated with a computer readable medium may be described in the general context of computer-executable instructions, such as, for example, program modules, which may be executed by a computer. Generally, program modules may include routines, programs, objects, components, data structures, etc. that perform particular tasks or implement particular abstract data types. The disclosed subject matter may also be practiced in distributed computing environments wherein tasks are performed by remote processing devices that are linked through a communications network. In a distributed computing environment, program modules may be located in local and/or remote computer storage media including memory storage devices.

The detailed description set forth herein in connection with the appended drawings is intended as a description of exemplary embodiments in which the presently disclosed subject matter may be practiced. The term “exemplary” used throughout this description means “serving as an example, instance, or illustration,” and should not necessarily be construed as preferred or advantageous over other embodiments.

This detailed description of illustrative embodiments includes specific details for providing a thorough understanding of the presently disclosed subject matter. However, it will be apparent to those skilled in the art that the presently disclosed subject matter may be practiced without these specific details. In some instances, well-known structures and devices are shown in block diagram form in order to avoid obscuring the concepts of the presently disclosed method and system.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein, but is to be accorded the widest scope consistent with the principles and novel features disclosed herein. It is contemplated that additional embodiments are within the spirit and true scope of the disclosed subject matter.

What is claimed is:

1. A method for simulation and evaluation of human physiological movements, comprising the steps of:
operating a three-dimensional video camera capable of communicating video images of a person recorded by said three-dimensional camera as a stream of video data;
operating at least one processor in association with said three-dimensional video camera for receiving from said three-dimensional video camera said stream of video data;
storing in at least one memory associated with said at least one processor computer code instructions comprising program instructions of data collection node instructions and skeletal joint measurement and condition reporting instructions, wherein said at least one memory and the computer code instructions operate in association with said at least one processor for performing the steps of:
recording live video position data from said three-dimensional video camera, said live video position data comprising three-dimensional vector data corresponding to a plurality of skeletal joint locations of the person in an XYZ-coordinate space, said live video position data further comprising depth of image and motion data associated with said plurality of skeletal joint locations;
operating said at least one processor to process said data collection node instructions for generating and associating with at least a subset of said plurality of skeletal joint locations a plurality of data collection nodes, said plurality of data collection nodes representing XYZ-coordinate data sets associated with said at least a subset of said plurality of skeletal joint locations and forming a simulation of human physiological measurement, analysis, and diagnosis an; and
storing in said at least one memory a baseline set of skeletal joint locations using said plurality of data collection nodes forming a simulation of human physiological movement using said data collection node instructions, said baseline set of skeletal joint locations associating with a predetermined set of baseline positions and movements of the person;
displaying on a computer display monitor associated with said at least one processor a simulation of human physiological movement display of said streams of video data of a person recorded by said three-dimensional video camera and said data collection nodes, and superimposing at least a subset of said data collections nodes over at least a subset of said skeletal joint locations within said display using said data collection node instructions;
recording and storing in said at least one memory a measured set of data collection nodes associated with a simulation of prescribed set of movements of the person by dynamically associating said data collection nodes with video images of said skeletal joint locations, as said skeletal joint locations change in response to the person performing said prescribed set of movements using said data collection node instructions;
automatically generating a plurality of skeletal joint measurements from said measured set of data collection nodes using said skeletal joint measurement and condition reporting instructions, said skeletal joint measurements comprising:
a plurality of Y reference measurements;
a plurality of hip reference measurements for determining a distance between skeletal joint locations of the person's hip joints and a skeletal joint location of the person's mid spine;
a pelvic drop/trunk lean measurement;
a plurality of two-joint angle measurements of a subset of said skeletal joint locations;
a pelvic drop/trunk lean measurement;
a plurality of three-joint angle measurements of a subset of said skeletal joint locations;
a hip flexion angle measurement; and
a knee valgus/varus measurement; and further
automatically storing in said at least one memory said plurality of skeletal joint measurements;
generating joint condition data values relating said plurality of skeletal joint measurements to a predetermined set of stored skeletal data values for identifying ones of said plurality of skeletal joint measurements corresponding to various conditions of said person at said skeletal joint locations during the performance of said prescribed set of movements; and
displaying said joint condition data values on said computer display monitor in association with said displaying of said stream of video data as recorded by said three-dimensional video camera.

2. The method of claim 1, further comprising the step of recording said skeletal location data wherein said prescribed set of movements may result in injury to the person's bodily due to lower flexibility and strength than a predetermined threshold.

3. The method of claim 1, wherein said step of operating at least one in association with said three-dimensional video camera for receiving from said three-dimensional video camera said stream of video data, further comprises the step of presenting said stream of video data as a stream of two-dimensional video from a color video camera combined with simultaneously generated stream of depth sensor measurement.

4. The method of claim 1, wherein said data collection nodes and said skeletal join locations correspond to a predetermined set of approximately 25 anatomical locations of the person.

5. A system for simulation and evaluation of human physiological movement, comprising:
at least one processor;
a three-dimensional video camera capable of communicating to said at least one processor video images of a person recorded by said three-dimensional camera as a stream of video data;
at least one memory associated with said at least one processor for storing computer code instructions comprising program instructions of data collection node instructions and skeletal joint measurement and condition reporting instructions for simulation of human physiological movement, wherein said at least one memory and the computer code instructions are configured with the at least one processor to perform the steps of:
said computer code instructions further comprising instructions for recording live video position data from said three-dimensional video camera, said live video position data comprising three-dimensional vector data corresponding to a plurality of skeletal joint locations of the person in an XYZ-coordinate space, said live video position data further comprising depth of image and motion data associated with said plurality of skeletal joint locations for simulation of human physiological movement;
said computer code instructions further comprising instructions for operating said at least one processor and said at least one memory for generating and associating with at least a subset of said plurality of skeletal joint locations a plurality of data collection nodes, said plurality of data collection nodes representing XYZ-coordinate data sets associated with said at least a subset of said plurality of skeletal joint locations for simulation of human physiological movement;
said computer code instructions further comprising instructions for storing in said at least one memory a baseline set of skeletal joint locations using said plurality of data collection nodes, said baseline set of skeletal joint locations associating with a predetermined set of baseline positions and movements of the person for simulation of human physiological movement;
a computer display monitor associated with said at least one processor for displaying said streams of video data of the person recorded by said three-dimensional video camera and said data collection nodes, and
said data collection node instructions further for superimposing at least a subset of said data collections nodes over at least a subset of said skeletal joint locations within said display;
said at least one memory for recording and storing a measured set of data collection nodes associated with a prescribed set of movements of the person by dynamically associating said data collection nodes with video images of said skeletal joint locations as said skeletal joint locations change in response to the person performing said prescribed set of movements;
a plurality of skeletal joint measurements automatically generated from said measured set of data collection nodes using said skeletal joint measurement and condition reporting instructions referencing said simulation of human physiological movement, said skeletal joint measurements comprising:
a plurality of Y reference measurements;
a plurality of hip reference measurements for determining a distance between skeletal joint locations of the person's hip joints and a skeletal joint location of the person's mid spine;
a pelvic drop/trunk lean measurement;
a plurality of two-joint angle measurements of a subset of said skeletal joint locations;
a pelvic drop/trunk lean measurement;
a plurality of three-joint angle measurements of a subset of said skeletal joint locations;
a hip flexion angle measurement; and
a knee valgus/varus measurement; and further
said at least one memory further configured for automatically storing said plurality of skeletal joint measurements;
joint condition data values relating said plurality of skeletal joint measurements to a predetermined set of stored skeletal data values for identifying ones of said plurality of skeletal joint measurements corresponding to various conditions of said person at said skeletal joint locations during the performance of said prescribed set of movements; and
said computer display monitor for displaying said joint condition data values in association with said displaying of said streams of video data as recorded by said three-dimensional video camera.

6. The system for simulation and evaluation of human physiological movement of claim 5, wherein said computer code instructions further comprise instructions for recording said skeletal location data wherein said prescribed set of movements may result in injury to the person's bodily due to lower flexibility and strength than a predetermined threshold.

7. The system for simulation and evaluation of human physiological movement of claim 5, wherein said computer code instructions further comprise instructions for operating at least one in association with said three-dimensional video camera for receiving from said three-dimensional video camera said stream of video data, further comprises the step of presenting said stream of video data as a stream of two-dimensional video from a color video camera combined with simultaneously generated stream of depth sensor measurement.

8. The system for simulation and evaluation of human physiological movement system of claim 5, wherein said computer code instructions further comprise instructions for identifying said data collection nodes and said skeletal join locations to correspond with a predetermined set of approximately 25 anatomical locations of the person.

8. A system for simulation and evaluation of human physiological movement, comprising:
at least one processor;
a three-dimensional video camera capable of communicating to said at least one processor video images of a person recorded by said three-dimensional camera as a stream of video data;
at least one memory associated with said at least one processor for storing computer code instructions comprising program instructions of data collection node instructions and skeletal joint measurement and condition reporting instructions, wherein said at least one memory and the computer code instructions are configured with the at least one processor to perform the steps of:
said computer code instructions further comprising instructions for recording live video position data from said three-dimensional video camera, said live video position data comprising three-dimensional vector data corresponding to a plurality of skeletal joint locations of the person in an XYZ-coordinate space, said live video position data further comprising depth of image and motion data associated with said plurality of skeletal joint locations;
said computer code instructions further comprising instructions for operating said at least one processor and said at least one memory for generating and associating with at least a subset of said plurality of skeletal joint locations a plurality of data collection nodes, said plurality of data collection nodes representing XYZ-coordinate data sets associated with said at least a subset of said plurality of skeletal joint locations;
said computer code instructions further comprising instructions for storing in said at least one memory a baseline set of skeletal joint locations using said plurality of data collection nodes, said baseline set of skeletal joint locations associating with a predetermined set of baseline positions and movements of the person;
a computer display monitor associated with said at least one processor for displaying said streams of video data of the person recorded by said three-dimensional video camera and said data collection nodes, and
said data collection node instructions further for superimposing at least a subset of said data collections nodes over at least a subset of said skeletal joint locations within said display;
said at least one memory for recording and storing a measured set of data collection nodes associated with a prescribed set of movements of the person by dynamically associating said data collection nodes with video images of said skeletal joint locations as said skeletal joint locations change in response to the person performing said prescribed set of movements
wherein said prescribed set of movements aid in the demonstration of limitations in the person's bodily flexibility and strength;
a plurality of skeletal joint measurements automatically generated from said measured set of data collection nodes using said skeletal joint measurement and condition reporting instructions, said skeletal joint measurements comprising:
a plurality of Y reference measurements;
a plurality of hip reference measurements for determining a distance between skeletal joint locations of the person's hip joints and a skeletal joint location of the person's mid spine;
a pelvic drop/trunk lean measurement;
a plurality of two-joint angle measurements of a subset of said skeletal joint locations;
a pelvic drop/trunk lean measurement;
a plurality of three-joint angle measurements of a subset of said skeletal joint locations;
a hip flexion angle measurement; and
a knee valgus/varus measurement; and further
said at least one memory further configured for automatically storing said plurality of skeletal joint measurements;
joint condition data values relating said plurality of skeletal joint measurements to a predetermined set of stored skeletal data values for identifying ones of said plurality of skeletal joint measurements corresponding to various conditions of said person at said skeletal joint locations during the performance of said prescribed set of movements; and
said computer display monitor for displaying said joint condition data values in association with said displaying of said streams of video data as recorded by said three-dimensional video camera.

9. The video-based system of claim 8, wherein said computer code instructions further comprise instructions for recording said skeletal location data wherein said prescribed set of movements may result in injury to the person's bodily due to lower flexibility and strength than a predetermined threshold.

10. The video-based system of claim 8, wherein said computer code instructions further comprise instructions for operating at least one in association with said three-dimensional video camera for receiving from said three-dimensional video camera said stream of video data, further comprises the step of presenting said stream of video data as a stream of two-dimensional video from a color video camera combined with simultaneously generated stream of depth sensor measurement.

11. The video-based system of claim 8, wherein said computer code instructions further comprise instructions for identifying said data collection nodes and said skeletal join locations to correspond with a predetermined set of approximately 25 anatomical locations of the person.
